(19) United States
(12) Patent Application Publication
(10) Pub. No.: US 2009/0312437 A1
(43) Pub. Date: Dec. 17, 2009

(54) Anthraquinones and Analogs from Rhuem palmatum for Treatment of Estrogen Receptor Beta-Mediated Conditions

(75) Inventor: Isaac Cohen, Pledmont, CA (US)

Correspondence Address: WILSON, SONSINI, GOODRICH & ROSATI, 650 PAGE MILL ROAD, PALO ALTO, CA, 94304-1050, US

(73) Assignee: Bionovo, Inc., A Delaware Corporation

(21) Appl. No.: 12/479,607

(22) Filed: Jun. 5, 2009

Related U.S. Application Data
(60) Provisional application No. 61/059,686, filed on Jun. 6, 2008.

(51) Int. Cl. A61K 31/122; C12N 5/02; C12S 3/00; C07C 50/34

(52) U.S. Cl. 514/680; 435/375; 435/267; 552/261; 552/262

(57) ABSTRACT

Compositions derived from Rheum palmatum are provided. Also provided are methods of using said extracts to induce apoptosis in specific cells, especially in a human. Provided as well are uses of the extracts of Rheum palmatum for the preparation of a medicament for the selective induction of apoptosis

### CROSS-REFERENCE

This application claims the benefit of U.S. Provisional Application No. 61/059,686, filed, Jun. 6, 2008, which is incorporated herein by reference in its entirety.

### BACKGROUND OF THE INVENTION

Hormone replacement therapy (HRT) has been used successfully to treat a variety of conditions, such as osteoporosis, increased risk of cardiovascular disease in post-menopausal women and climacteric symptoms, such as hot flashes, decreased libido and depression. However, HRT with estradiol (E₂), either alone or in combination with progestin, can lead to undesirable effects. In fact, a recent Women's Health Initiative (WHI) study was abruptly halted when preliminary results showed that HRT was associated with a 35% increased risk of breast cancer.

Breast cancer can be treated or prevented by using a so-called selective estrogen receptor modulator (SERM), such as tamoxifen. (Before the approval of tamoxifen, breast cancer treatment of pre-menopausal women often included removing the ovaries in order to reduce the cancer-stimulating effect of estrogen.) Tamoxifen appears to selectively block the cancer-inducing effects of estrogen in breast tissues of pre-menopausal women. Another SERM, raloxifene, has been approved for treatment of osteoporosis as an alternative to estrogen replacement. In addition to selectively inducing estrogenic effects in bone tissue, long-term administration of raloxifene was also shown to be associated with reduction in the rate of breast cancer in the Multiple Outcomes of Raloxifene Evaluation (MORE) study.

While SERMs such as tamoxifen and raloxifene provide selective reduction in estrogen's cancer-inducing effects in the breast, they are not without their risks. For example both tamoxifen and raloxifene therapy have been associated with increased incidence of hot flushes, and tamoxifen therapy has been shown to increase the risk of uterine (endometrial) cancer.

Despite the success of estrogen replacement therapy in treating osteoporosis, coronary heart disease and climacteric symptoms, and of SERMs like tamoxifen and raloxifene in treating breast cancer and osteoporosis, there remains a need for compositions having estrogenic properties. Additionally, given the increasing cost of producing drug compounds, there is a need for additional estrogenic compositions that may be obtained from natural sources.

### SUMMARY OF THE INVENTION

The inventor has determined that compositions comprising a compound or compounds derived from an extract of Rheum palmatum (especially the rhizome thereof) have ERβ-inhibitory effects. In particular compositions comprising anthraquinones or their analogs from Rheum palmatum extracts selectively induce apoptosis in ERα-negative cancerous cells, while ERα-positive cells are resistant to the cytotoxic effects of the extracts. Thus, extracts described herein, i.e. extracts of Rheum palmatum, and compositions comprising such extracts, are selective selective estrogenic agents useful for the treatment of disease states characterized by ERα-negative hyperproliferation of cells, such as ERα-negative cancer and benign hyperplastic disorders such as BPH and restenosis.

Some embodiments described herein provide a pharmaceutical composition, comprising an amount of at least one isolated and purified compound of formula I or 6, wherein formula I is:

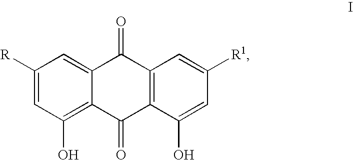

where R is H or OH; and R¹ is C₁-C₄ alkyl (methyl, ethyl, isopropyl, n-propyl, isobutyl, s-butyl, n-butyl or t-butyl) or CH₂OH; and
formula 6 is: octahydroxyanthraquinone (formula 6):

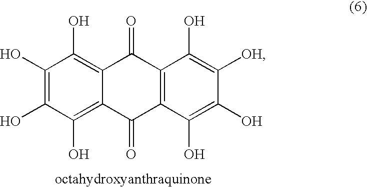

wherein the amount is sufficient to modulate estrogen receptor beta (ERβ) in a multicellular organism. In some embodiments, at least one compound is selected from compounds 1, 2 and/or 3 of formula II:

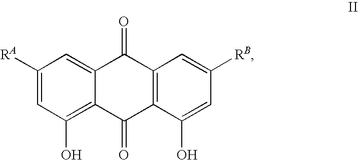

wherein either (1) R^{A} is OH and R^{B} is CH₃; (2) R^{A} is H and R^{B} is CH₂OH; (3) R^{A} is H and R^{B} is CH₃; and the compound of formula (6) is:

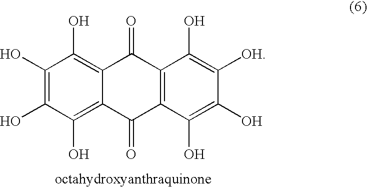

In some embodiments, the composition comprises two or more of (1), (2), (3) and/or (6). In some embodiments, the composition comprises three or more of (1), (2), (3) and/or (6). In some embodiments, the composition comprises all four of (1), (2), (3) and/or (6). In some embodiments, the composition is substantially free of one or both of rhein and frangulin A; in some embodiments, the composition is free of both rhein and frangulin A. In some embodiments, the composition comprises both of (1) and (6). In some embodiments, the composition comprises both of (2) and (6). In some embodiments, the composition comprises both of (3) and (6). In some embodiments, the composition comprises (1), (2) and (6). In some embodiments, the composition comprises (1), (3) and (6). In some embodiments, the composition comprises (2), (3) and (6). In some embodiments, the composition comprises (1), (2), (3) and (6). In some embodiments, the composition comprises (1) and (3). In some embodiments, the composition comprises (1) and (2). In some embodiments, the composition comprises (1), (2) and (3). In some embodiments, the composition comprises (2) and (3). In some embodiments, the composition is for use in the manufacture of a medicament. In some embodiments the medicament possesses a selective estrogen receptor beta-agonistic effect. In some embodiments, the medicament possesses a selective estrogen receptor beta-agonistic effect. In some embodiments, the estrogenic effect is at least one effect selected from the group consisting of: treating or preventing at least one climacteric symptom; treating or preventing osteoporosis; treating or preventing uterine cancer; treating or preventing breast cancer; treating or preventing cervical cancer; treating or preventing cancer of the ovary; and treating or preventing cardiovascular disease. In some embodiments, the estrogenic effect includes treating or preventing at least one climacteric symptom selected from the group consisting of treating or preventing hot flashes, insomnia, vaginal dryness, decreased libido, urinary incontinence and depression. In some embodiments the estrogenic effect includes treating or preventing osteoporosis. In some embodiments the estrogenic effect includes treating or preventing hot flashes. In some embodiments the estrogenic effect includes treating or preventing uterine cancer or breast cancer. In some embodiments the estrogenic effect does not include increasing the risk of mammary hyperplasia, mammary tumor, uterine hyperplasia, uterine tumor, cervical hyperplasia, cervical tumor, ovarian hyperplasia, ovarian tumor, fallopian tube hyperplasia, fallopian tube tumor. In some embodiments the estrogenic effect includes decreasing the risk of mammary hyperplasia, mammary tumor, uterine hyperplasia, uterine tumor, cervical hyperplasia, cervical tumor, ovarian hyperplasia, ovarian tumor, fallopian tube hyperplasia, fallopian tube tumor. In some embodiments there is provided the use of compositions described herein for the preparation of a medicament. Some embodiments provide a method of preparing a unit dosage form of a medicament, comprising combining a composition as described herein with at least one additional pharmaceutically acceptable ingredient, wherein the additional ingredient is active or inert.

Some embodiments described herein provide a method of eliciting an estrogenic effect, comprising administering to a subject an estrogenically effective amount of a composition comprising at least -one isolated and purified compound of formula I or 6:

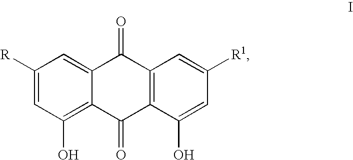

where R is H or OH; and
R¹ is C₁-C₄ alkyl (methyl, ethyl, isopropyl, n-propyl, isobutyl, n-butyl, s-butyl or t-butyl) or CH₂OH; and compound 6 has the formula:

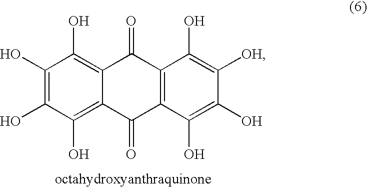

wherein the amount administered is sufficient to modulate (e.g. agonize) estrogen receptor beta (ERβ) in a multicellular organism. In some embodiments, the compositions comprise at least one compound is selected from compounds 1-3 or 6, wherein the compounds 1-3 are of formula II:

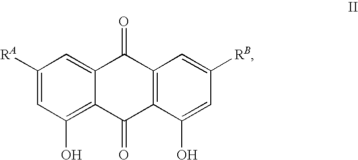

wherein either (1) R^{A} is OH and R^{B} is CH₃; (2) R^{A} is H and R^{B} is CH₂OH; (3) R^{A} is H and R^{B} is CH₃; or compound 6:

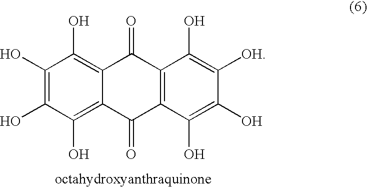

In some embodiments, the composition comprises two or more of (1), (2), (3) and/or (6). In some embodiments, the composition comprises three or more of (1), (2), (3) and/or (6). In some embodiments, the composition comprises all four of (1), (2), (3) and/or (6). In some embodiments, the composition is substantially free of one or both of rhein and frangulin A; in some embodiments, the composition is free of both rhein and frangulin A. In some embodiments, the composition comprises both of (1) and (6). In some embodiments, the composition comprises both of (2) and (6). In some embodiments, the composition comprises both of (3) and (6). In some embodiments, the composition comprises (1), (2) and (6). In some embodiments, the composition comprises (1), (3) and (6). In some embodiments, the composition comprises (2), (3) and (6). In some embodiments, the composition comprises (1), (2), (3) and (6). In some embodiments, the composition comprises (1) and (3). In some embodiments, the composition comprises (1) and (2). In some embodiments, the composition comprises (1), (2) and (3). In some embodiments, the composition comprises (2) and (3). In some embodiments, the estrogenic effect is at least one effect selected from the group consisting of: treating or preventing at least one climacteric symptom; treating or preventing osteoporosis; treating or preventing uterine cancer; treating or preventing breast cancer; treating or preventing cervical cancer; treating or preventing cancer of the ovary; and treating or preventing cardiovascular disease. In some embodiments, the estrogenic effect includes treating or preventing at least one climacteric symptom selected from the group consisting of treating or preventing hot flashes, insomnia, vaginal dryness, decreased libido, urinary incontinence and depression. In some embodiments, the estrogenic effect includes treating or preventing osteoporosis. In some embodiments, the estrogenic effect includes treating or preventing hot flashes. In some embodiments, the estrogenic effect includes treating or preventing uterine cancer or breast cancer. In some embodiments, the estrogenic effect does not include increasing the risk of mammary hyperplasia, mammary tumor, uterine hyperplasia, uterine tumor, cervical hyperplasia, cervical tumor, ovarian hyperplasia, ovarian tumor, fallopian tube hyperplasia, fallopian tube tumor. In some embodiments, the estrogenic effect includes decreasing the risk of mammary hyperplasia, mammary tumor, uterine hyperplasia, uterine tumor, cervical hyperplasia, cervical tumor, ovarian hyperplasia, ovarian tumor, fallopian tube hyperplasia, fallopian tube tumor.

Some embodiments described herein provide a method of activating a gene under control of an estrogen response element, comprising administering to a cell having an estrogen response element operatively linked to the gene and an estrogen receptor an amount of a composition sufficient to activate said gene, wherein the composition at least one isolated and purified compound of formula I or 6:

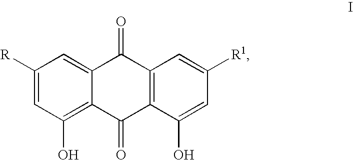

where R is H or OH; and
R¹ is C₁-C₄ alkyl (methyl, ethyl, isopropyl, n-propyl, isobutyl, n-butyl, s-butyl or t-butyl) or CH₂OH; and compound 6 has the formula:

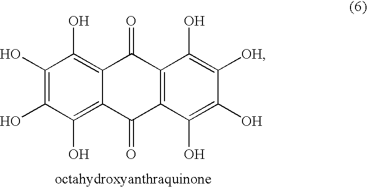

wherein the amount administered is sufficient to modulate (e.g. agonize) estrogen receptor beta (ERβ) in a multicellular organism. In some embodiments, the compositions comprise at least one compound is selected from compounds 1-3 or 6, wherein the compounds 1-3 are of formula II:

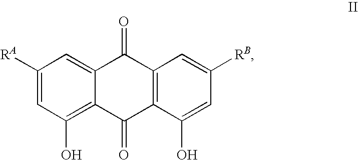

wherein either (1) R^{A} is OH and R^{B} is CH₃; (2) R^{A} is H and R^{B} is CH₂OH; (3) R^{A} is H and R^{B} is CH₃; or
compound 6:

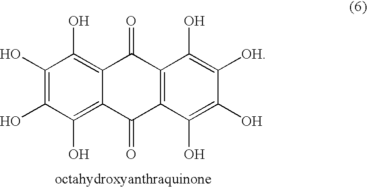

In some embodiments, the composition comprises two or more of (1), (2), (3) and/or (6). In some embodiments, the composition comprises three or more of (1), (2), (3) and/or (6). In some embodiments, the composition comprises all four of (1), (2), (3) and/or (6). In some embodiments, the composition is substantially free of one or both of rhein and frangulin A; in some embodiments, the composition is free of both rhein and frangulin A. In some embodiments, the composition comprises both of (1) and (6). In some embodiments, the composition comprises both of (2) and (6). In some embodiments, the composition comprises both of (3) and (6). In some embodiments, the composition comprises (1), (2) and (6). In some embodiments, the composition comprises (1), (3) and (6). In some embodiments, the composition comprises (2), (3) and (6). In some embodiments, the composition comprises (1), (2), (3) and (6). In some embodiments, the composition comprises (1) and (3). In some embodiments, the composition comprises (1) and (2). In some embodiments, the composition comprises (1), (2) and (3). In some embodiments, the composition comprises (2) and (3). In some embodiments, the cell having an estrogen response element operative linked to a gene and an estrogen receptor is in vitro. In some embodiments, the cell is in vivo. In some embodiments, the cell is in an ERα+ breast tissue. In some embodiments, the cell is in an ERβ+ breast tissue. In some embodiments, the cell is in an ERα/ERβ+ breast tissue. In some embodiments, the estrogen response element is expressed in a transformed cell. In some embodiments, the estrogen response element and the estrogen receptor are expressed in a transformed cell. In some embodiments, the estrogen response element is heterologously expressed in the cell. In some embodiments, the estrogen response element and the estrogen receptor are heterologously expressed in the cell. In some embodiments, the cell is selected from the group consisting of a U937, a U2OS, a MDA-MB-435 and a MCF-7 cell transformed with an ERE-controlled gene. In some embodiments, the cell expresses ERα. In some embodiments, the cell expresses ERβ. In some embodiments, the ERE-controlled gene is ERE-tk-Luc.

Some embodiments described herein further provide a method of repressing expression of a TNF RE-controlled gene, comprising administering to a cell comprising a gene under control of a TNF response element and an estrogen receptor an amount of a composition as described herein effective to repress said TNF RE-controlled gene, said composition comprising at least one isolated and purified compound of formula I or 6:

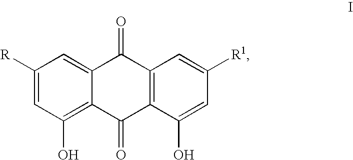

wherein R is H or OH; and R¹ is C₁-C₄ alkyl (methyl, ethyl, isopropyl, n-propyl, isobutyl, n-butyl, s-butyl or t-butyl) or CH₂OH; and compound 6 has the formula:

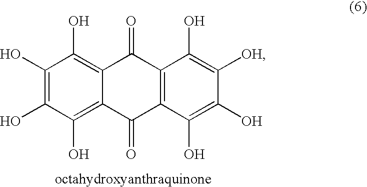

wherein the amount administered is sufficient to modulate (e.g. agonize) estrogen receptor beta (ERβ) in a multicellular organism. In some embodiments, the compositions comprise at least one compound is selected from compounds 1-3 and/or 6, wherein the compounds 1-3 are of formula II:

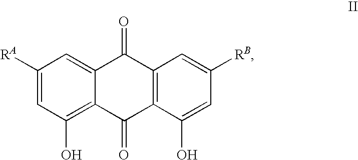

wherein either (1) R^{A} is OH and R^{B} is CH₃; (2) R^{A} is H and R^{B} is CH₂OH; (3) R^{A} is H and R^{B} is CH₃; and
compound 6 is:

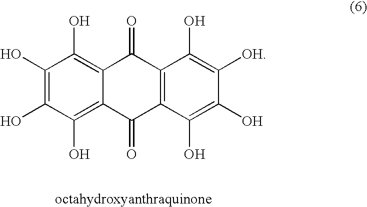

In some embodiments, the composition comprises two or more of (1), (2), (3) and/or (6). In some embodiments, the composition comprises three or more of (1), (2), (3) and/or (6). In some embodiments, the composition comprises all four of (1), (2), (3) and/or (6). In some embodiments, the composition is substantially free of one or both of rhein and frangulin A; in some embodiments, the composition is free of both rhein and frangulin A. In some embodiments, the composition comprises both of (1) and (6). In some embodiments, the composition comprises both of (2) and (6). In some embodiments, the composition comprises both of (3) and (6). In some embodiments, the composition comprises (1), (2) and (6). In some embodiments, the composition comprises (1), (3) and (6). In some embodiments, the composition comprises (2), (3) and (6). In some embodiments, the composition comprises (1), (2), (3) and (6). In some embodiments, the composition comprises (1) and (3). In some embodiments, the composition comprises (1) and (2). In some embodiments, the composition comprises (1), (2) and (3). In some embodiments, the composition comprises (2) and (3). In some embodiments, the TNF RE-controlled gene is TNF-α. In some embodiments, the TNF RE-controlled gene is TNF RE-Luc. In some embodiments, the cell is in vitro. In some embodiments, the cell is in vivo. In some embodiments, the cell is in an ER+ breast tissue. In some embodiments, the cell is in an ERα+ breast tissue. In some embodiments, the cell is in an ERβ+ breast tissue. In some embodiments, the TNF response element is endogenously expressed in the cell. In some embodiments, the TNF response element and the estrogen receptor are endogenously expressed in the cell. In some embodiments, the TNF response element is heterologously expressed in the cell. In some embodiments, the TNF response element and the estrogen receptor are heterologously expressed in the cell. In some embodiments, the cell contains an estrogen receptor gene, is transformed with a TNF response element-controlled gene, and is selected from the group consisting of a U937, a U2OS, a MDA-MB-435 and a MCF-7 cell. In some embodiments, the estrogen receptor gene is a gene expressing ERα. In some embodiments, the estrogen receptor gene is a gene expressing ERβ.

Some embodiments described herein provide a process of isolating emodin from Rheum palmatum, comprising: (a) contacting optionally wholly or partially comminuted rhizome of Rheum palmatum with aqueous methanol; (b) separating the rhizome from the aqueous methanol to form an aqueous methanol extract; (c) evaporating methanol from the aqueous methanol extract to form a concentrate; (d) adding water to the concentrate to form an aqueous slurry; (e) contacting the aqueous slurry with hexane and separating the hexane from the aqueous slurry; (f) contacting the aqueous slurry with ethyl acetate; (g) separating the ethyl acetate from the aqueous slurry; (h) applying the ethyl acetate to a solid phase extraction substrate; (i) serially eluting the extraction substrate with serial aliquots of elution solvents (A)-(D): (A) aqueous ammonium acetate, (B) aqueous ammonium acetate and acetonitrile in a ratio of about 4:1 to about 2.5:1; (C) aqueous ammonium acetate and acetonitrile in a ratio of about 0.9:1 to about 1.1:1; and (D) aqueous ammonium acetate and acetonitrile in a ratio of about 1:2.5 to about 1:4; (j) collecting an eluate of (D) and applying the collected eluate to a silica gel; (k) contacting the silica gel with a mixture of hexane, ethyl acetate and trifluoroacetic acid; and (l) collecting a fraction containing emodin from the silica gel. In some embodiments, one or more of the following applies: (i) elution solvent (B) contains about 10 mM ammonium acetate (aqueous) and acetonitrile in a ratio of about 3:1 (v/v); (ii) elution solvent (C) contains about 10 mM ammonium acetate (aqueous) and acetonitrile in a ratio of about 1:1 (v/v); (iii) elution solvent (D) contains about 10 mM ammonium acetate (aqueous) and acetonitrile in a ratio of about 1:3 (v/v); (iv) the solid phase extraction substrate in (h) is a reverse phase extraction substrate in a column or cartridge; (v) the silica gel in (j) is on a silica gel thin layer chromatography (TLC) plate; and/or (vi) the mixture of hexane, ethyl acetate and trifluoroacetic acid in (k) is in a ratio of about 8:2:0.1.

Some embodiments described herein provide a process of isolating aloe-emodin from Rheum palmatum, comprising: (a) contacting optionally wholly or partially comminuted rhizome of Rheum palmatum with aqueous methanol; (b) separating the rhizome from the aqueous methanol to form an aqueous methanol extract; (c) evaporating methanol from the aqueous methanol extract to form a concentrate; (d) adding water to the concentrate to form an aqueous slurry; (e) contacting the aqueous slurry with hexane and separating the hexane from the aqueous slurry; (f) contacting the aqueous slurry with ethyl acetate; (g) separating the ethyl acetate from the aqueous slurry; (h) applying the ethyl acetate to a solid phase extraction substrate; (i) eluting the extraction substrate with serial aliquots of extraction solvents (A)-(D): (A) aqueous ammonium acetate, (B) aqueous ammonium acetate and acetonitrile in a ratio of about 4:1 to about 2.5:1; (C) aqueous ammonium acetate and acetonitrile in a ratio of about 0.9:1 to about 1.1:1; and (D) aqueous ammonium acetate and acetonitrile in a ratio of about 1:2.5 to about 1:4; (j) collecting an eluate of (D) and applying the collected eluate to a silica gel; (k) contacting the silica gel with a mixture of hexane, ethyl acetate and trifluoroacetic acid in a ratio of about 8:2:0.1; and (l) collecting a fraction containing aloe-emodin from the silica gel. In some embodiments, one or more of the following applies: (i) elution solvent (B) contains about 10 mM ammonium acetate (aqueous) and acetonitrile in a ratio of about 3:1 (v/v); (ii) elution solvent (C) contains about 10 mM ammonium acetate (aqueous) and acetonitrile in a ratio of about 1:1 (v/v); (iii) elution solvent (D) contains about 10 mM ammonium acetate (aqueous) and acetonitrile in a ratio of about 1:3 (v/v); (iv) the solid phase extraction substrate in (h) is a reverse phase extraction substrate in a column or cartridge; (v) the silica gel in (j) is on a silica gel thin layer chromatography (TLC) plate; and/or (vi) the mixture of hexane, ethyl acetate and trifluoroacetic acid in (k) is in a ratio of about 8:2:0.1.

Some embodiments described herein provide a process of isolating chrysophanol from Rheum palmatum, comprising: (a) contacting optionally wholly or partially comminuted rhizome of Rheum palmatum with aqueous methanol; (b) separating the rhizome from the aqueous methanol to form an aqueous methanol extract; (c) evaporating methanol from the aqueous methanol extract to form a concentrate; (d) adding water to the concentrate to form an aqueous slurry; (e) contacting the aqueous slurry with hexane and separating the hexane from the aqueous slurry; (f) contacting the aqueous slurry with ethyl acetate; (g) separating the ethyl acetate from the aqueous slurry; (h) applying the ethyl acetate to a solid phase extraction substrate; (i) eluting the extraction substrate with serial aliquots of extraction solvents (A)-(D): (A) aqueous ammonium acetate, (B) aqueous ammonium acetate and acetonitrile in a ratio of about 4:1 to about 2.5:1; (C) aqueous ammonium acetate and acetonitrile in a ratio of about 0.9:1 to about 1.1:1; and (D) aqueous ammonium acetate and acetonitrile in a ratio of about 1:2.5 to about 1:4; (j) collecting an eluate of (D) and applying the collected eluate to a silica gel; (k) contacting the silica gel with a mixture of hexane, ethyl acetate and trifluoroacetic acid in a ratio of about 8:2:0.1; and (l) collecting a fraction from the silica gel containing chrysophanol. In some embodiments, one or more of the following applies: (i) elution solvent (B) contains about 10 mM ammonium acetate (aqueous) and acetonitrile in a ratio of about 3:1 (v/v); (ii) elution solvent (C) contains about 10 mM ammonium acetate (aqueous) and acetonitrile in a ratio of about 1:1 (v/v); (iii) elution solvent (D) contains about 10 mM ammonium acetate (aqueous) and acetonitrile in a ratio of about 1:3 (v/v); (iv) the solid phase extraction substrate in (h) is a reverse phase extraction substrate in a column or cartridge; (v) the silica gel in (j) is on a silica gel thin layer chromatography (TLC) plate; and/or (vi) the mixture of hexane, ethyl acetate and trifluoroacetic acid in (k) is in a ratio of about 8:2:0.1.

Some embodiments described herein provide a process of isolating octahydroxyanthraquinone from Rheum palmatum, comprising: (a) contacting optionally wholly or partially comminuted rhizome of Rheum palmatum with aqueous methanol; (b) separating the rhizome from the aqueous methanol to form an aqueous methanol extract; (c) evaporating methanol from the aqueous methanol extract to form a concentrate; (d) adding water to the concentrate to form an aqueous slurry; (e) contacting the aqueous slurry with hexane and separating the hexane from the aqueous slurry; (f) contacting the aqueous slurry with ethyl acetate; (g) separating the ethyl acetate from the aqueous slurry; (h) applying the ethyl acetate to a solid phase extraction cartridge; (i) eluting the extraction cartridge with serial aliquots of (A) aqueous ammonium acetate, (B) aqueous ammonium acetate and acetonitrile in a ratio of about 4:1 to about 2.5:1; and (C) aqueous ammonium acetate and acetonitrile in a ratio of about 0.9:1 to about 1.1:1; (j) collecting an eluate of (C) and applying the collected eluate to a resin separation substrate, eluting with a lower alcohol (such as ethanol or methanol) and collecting an octahydroxyanthraquinone-containing fraction; (1) applying the collected fraction to a reverse phase separation substrate and fractionating with a mixture of ammonium acetate and acetonitrile in a ratio of about 5:5 to about 7:3; (m) collecting a fraction containing octahydroxyanthraquinone and applying the obtained fraction to a silica gel; (n) collecting a fraction from the developed silica gel containing octahydroxyanthraquinone. In some embodiments, one or more of the following applies (i) elution solvent (B) contains about 10 mM ammonium acetate (aqueous) and acetonitrile in a ratio of about 3:1 (v/v); (ii) elution solvent (C) contains about 10 mM ammonium acetate (aqueous) and acetonitrile in a ratio of about 1:1 (v/v); (iii) the solid phase extraction substrate in (h) is a reverse phase extraction substrate in a column or cartridge; (iv) the silica gel in (j) is on a silica gel thin layer chromatography (TLC) plate; and/or (v) the mixture of hexane, ethyl acetate and trifluoroacetic acid in (k) is in a ratio of about 8:2:0.1.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows overlaid chromatograms of anthraquinones, emodin (1), aloe-emodin (2), chrysophanol (3), rhein (4), frangulin A (5), and octahydroxyanthraquinone (6).

FIG. 2 shows that emodin (1) has a selective agonistic effect on ERβ, but not ERα.

FIG. 3 shows the ERβ-selective estrogenic effect of octahydroxyanthraquinone (6); the compound 6 has no effect on ERα.

FIG. 4 shows the effect of octahydroxyanthraquinone (6) on uterine proliferation as compared to control (vehicle) and estradiol (E₂). Estradiol stimulates uterine proliferation, whereas 6 inhibits uterine proliferation as compared to control

FIG. 5A depicts pictures of kidney capsule grafts. The pictures show the effect of octahydroxyanthraquinone (6) on tumor formation in nude mice. Control=A, octahydroxyanthraquinone (6)=B, estradiol=C. As can be seen, octahydroxyanthraquinone suppresses tumor proliferation in murine kidney capsule grafts as compared to control, whereas E₂ stimulates tumor proliferation; FIG. 5B shows the results of the experiments graphically. MCF-7 tumor weight after 28 days was much greater in estradiol-treated nude mice bearing kidney capsule xenografts than control or octahydroxyanthraquinone (“octa”) treated mice.

FIG. 6 is a graph comparing the ERβ-activation of ERE-tk-luc in the presence of emodin with that of ERα-activation of ERE-tk-luc.

FIG. 7 is a graph comparing emodin TNF-α activation of TNF-RE in the presence of ERβ with emodin TNF-α activation of the TNF-RE in the presence of ERβ.

FIG. 8 shows ERβ-activation of ERE-tk-luc in the presence of emodin+control, emodin+raloxifene, emodin+tamoxifen and emodin+estradiol. As can be seen emodin alone and emodin plus estradiol stimulate expression of the ERE-tk-luc by about 4× and 6×, respectively, whereas the activation in the presence of emodin and either raloxifene or tamoxifen is actually repressed.

FIG. 9 shows the emodin binding curves for ERβ and ERα.

FIG. 10 is a graph comparing the ERβ-activation of ERE-tk-luc in the presence of aloe-emodin with that of ERα-activation of ERE-tk-luc.

FIG. 11 is a graph comparing aloe-emodin TNF-α activation of TNF-RE in the presence of ERβ with aloe-emodin TNF-α activation of the TNF-RE in the presence of ERβ.

FIG. 12 shows ERβ-activation of ERE-tk-luc in the presence of aloe-emodin+control, aloe-emodin+raloxifene, aloe-emodin+tamoxifen and aloe-emodin+estradiol. As can be seen aloe-emodin alone and emodin plus estradiol stimulate expression of the ERE-tk-luc by about 4× and 6×, respectively, whereas the activation in the presence of emodin and either raloxifene or tamoxifen is actually repressed.

FIG. 13 shows the aloe-emodin binding curves for ERβ and ERα.

### DETAILED DESCRIPTION OF THE INVENTION

Disclosed herein are pharmaceutical compositions comprising certain compounds of formula I or 6:

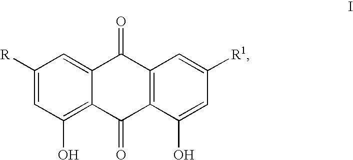

wherein R is H or OH; and
R¹ is C₁-C₄ alkyl (methyl, ethyl, isopropyl, n-propyl, isobutyl, n-butyl, s-butyl or t-butyl) or CH₂OH; and compound 6 has the formula:

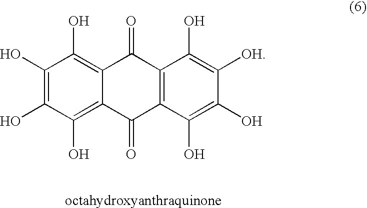

It has been discovered that such compositions containing one or more compounds of formula I or 6 comprise ERβ-selective estrogenic effects. Such estrogenic effects comprise prevention or treatment of estrogen receptor beta (ERβ) mediated diseases or conditions, such as one or more climacteric symptoms, estrogen receptor-mediated cancers (e.g. breast cancer, uterine cancer, ovary cancer, vaginal cancer, vulval cancer, cancer of the fallopian tubes, endometrial carcinoma and/or osteoporosis. Thus, in some embodiments, there are presented herein a method of treating a patient, comprising administering to the patient an estrogenically effective amount of a composition comprising 1 or more, 2 or more, 3 or more or all 4 of compounds 1, 2, 3 and or 6. In some embodiments, the method comprises administering a composition comprising at least compounds 1 and 6, at least compounds 2 and 6, at least compounds 3 and 6, at least compounds 1, 2 and 6, at least compounds 1, 3 and 6, at least compounds 1, 2, 3 and 6, at least compounds I and 3, at least compounds 1 and 2, at least compounds 1, 2 and 3, at least compounds 2, 3 and 6 or all four of compounds 1, 2, 3 and 6. As used herein, compound 1 is emodin (compound of formula I, wherein R is OH and R¹ is CH₃), compound 2 is aloe-emodin (compound of formula I, wherein R is H and R¹ is CH₂OH), compound 3 is chrysophanol (R is H and R¹ is CH₃) and compound 6 is octahydroxyanthraquinone.

Also described herein are novel methods of isolating compounds of formulae 1, 2, 3 and 6 from Rheum palmatum, especially from the rhizome of Rheum palmatum.

### Putative Targets and Modes of Action for Compounds 1, 2, 3 and 6

Breast neoplasms are the most common cancers diagnosed in women. In 2000, 184,000 new cases of breast cancer were diagnosed and 45,000 women died from breast cancer. Although the cause of breast cancer is probably multifactorial, there is compelling clinical, epidemiological and biological research that indicate estrogens promote breast cancer: (a) Hormone replacement therapy (HRT) is associated with a 35% increased risk of breast cancer by a meta-analysis of 51 studies; (b) Breast cancer can be prevented with tamoxifen or raloxifene, which bind to ERs and antagonize the actions of estrogens in breast cells; (c) Bilateral oophorectomy in premenopausal women with breast cancer leads to increased survival; (d) Greater exposure to estrogens (early menarche or late menopause, relative risk=1.3 and 1.5 to 2.0, respectively) increases the incidence of breast cancer; (e) Estrogens increase the proliferation of ER positive breast cancer cells; and (f) Estrogens increase the production of growth promoting genes, such as cyclin Dl, c-myc, and c-fos.

Approximately 60-70% of breast tumors contain estrogen receptors. For several decades, breast tumors have been analyzed for the presence of ERs. Approximately 70% of ER+ tumors are responsive to antiestrogen therapy. This observation has led to the notion that ER+ tumors have a better prognosis than ER negative tumors. However, the discovery of ERβ has complicated these interpretations and has raised some profound clinical questions. Understanding the role of ERα and ERβ is of paramount importance, because the current methods of determining whether tumors are ER+ uses an antibody that only detects ERα. Thus, most studies examining the effects ERs in breast tumors on clinical outcomes reflect the only ERα status. However, several recent studies have detected the presence of ERβ mRNA in human breast tumors. Most of the studies relied on RT-PCR to measure ERβ, because of the lack of specific and sensitive antibodies to ERβ. Dotzlaw et al. were the first to detect ERβ in breast tumor biopsies by RT-PCR. They found 70% of the breast tumors expressed ERβ and 90% expressed ERα. Furthermore, they demonstrated that several ER negative cell lines also express ERβ mRNA. These findings suggest that ERβ is highly expressed in breast tumors, and that both ERα and ERβ are often coexpressed in many tumors. In fact, some ER− tumors contain ERβ. Dotzlaw et al. also showed that ERβ mRNA is significantly lower in ER+/PR− (PR being progestin receptor) tumors compared to ER+/PR+ tumors. The authors suggested that this observation indicates that ERβ expression is associated with a poorer prognosis, because ER+/PR+ are more likely to respond to tamoxifen. Other studies suggest that the presence of ERβ confers a poor prognosis. Speirs et al. found that most breast tumors express ERβ mRNA alone or in combination with ERα mRNA. Those tumors that express both ERα and ERβ mRNA were associated with positive lymph nodes and tended to be characterized as higher grade tumors. Furthermore, increased ERβ expression occurs in MCF-10F cells treated with chemical carcinogens, suggesting that the expression of ERβ may contribute to the initiation and progression of breast cancer. Recently, Jensen et al. analyzed the expression of ERβ in 29 invasive breast tumors by immunohistochemistry (IHC). They found that ERβ expression was associated with an elevation of specific markers of cell proliferation, Ki67 and cyclin A. Moreover, the highest expression of these proliferation markers was present in ERα+/ERβ+ tumors. Although the number of ERα-/ERβ+cases were very small (n=7) the authors suggested that ERβ mediates cell proliferation in breast tumors. Speirs et al. also reported ERβ mRNA is significantly elevated in the tamoxifen-resistant tumors compared to tamoxifen-sensitive tumors.

In contrast, other studies indicate that the presence of ERβ confers a favorable prognosis. Iwao et al. demonstrated that ERα mRNA is up-regulated and ERβ mRNA is down-regulated as breast tumors progress from preinvasive to invasive tumors. Using IHC of frozen tumor sections Jarvinen et al. found that ERβ expression was associated with negative axillary node status, low grade, and low S-phase fraction. A study by Omoto et al. also found that ERβ positive tumors correlated with a better prognosis than ERβ negative tumors, because the disease-free survival rate was higher in tumors containing ERβ. ERβ expression also showed a strong association with the presence of progesterone receptors and well-differentiated breast tumors. It has also been reported that the levels of ERβ are highest in normal mammary tissue and that it decreases as tumors progress from pre-cancerous to cancerous lesions. These studies indicate that ERβ may function as a tumor suppressor and that the loss of ERβ promotes breast carcinogenesis. In a study by Mann et al. it was shown that the expression of ERβ in more than 10% of cancer cells was associated with better survival in women treated with tamoxifen. The aggregate of these studies indicates the presence of ERβ confers a favorable prognosis. Consistent with RT-PCR and IHC data is a report that showed that adenovirus-mediated expression of ERβ resulted in a ligand-independent inhibition of proliferation of the ER negative cell line, MDA-MB-231.

These results demonstrate that the role of ERβ in the pathogenesis and prognosis of breast cancer is unclear. Several reasons may explain the apparent discrepancy among these studies. First, there may be a poor correlation between ERβ mRNA and ERβ protein. This notion is consistent with the presence of ERβ mRNA in some ER negative cell lines that do not have detectable ERs by ligand binding assays. Second, the IHC studies used different commercially available ERβ antibodies that have been poorly characterized for specificity and sensitivity. Third, most of the conclusions have been based on a few breast cancer cases. Clearly, more studies are needed to clarify the role of ERα and ERβ in breast cancer.

Role of SERMs as adjuvant therapy and chemoprevention in breast cancer: Because estrogens promote the proliferation of breast cancer cells, several therapeutic approaches have been implemented to block this effect of estrogens on breast tumors. These strategies, including ovarian ablation, antiestrogens, gonadotropin releasing hormone analogs or aromatase inhibitors, work by either decreasing the production of estrogens or blocking the action of estrogens. All of these strategies non-selectively block the action of both ERα and ERβ. The most common approach used clinically to prevent and treat breast tumors are the selective estrogen receptor modulators (SERMs), tamoxifen and raloxifene.

Tamoxifen is a non-steroidal triphenylethylene derivative that is the prototype SERM, because it exhibits antagonistic action in some tissues, such as the breast, but has agonist actions in other tissues such as the endometrium and bone. Tamoxifen has been extensively studied for its clinical effectiveness as an adjuvant therapy to reduce the recurrences of breast tumors in women with estrogen receptor-positive breast cancer. Five years of tamoxifen therapy reduces the risk of recurrences by 42%, mortality from breast cancer by 22% and a second contralateral primary breast tumor. Approximately, ⅔ of ER positive breast tumors respond to tamoxifen, whereas very little evidence indicates that women with ER negative tumors benefit from adjuvant tamoxifen. Most recently, the U.S. Breast Cancer Prevention Trial (BCPT) demonstrated that tamoxifen reduces the risk of primary invasive breast cancer by 49% in women considered to be at high risk for breast cancer. These studies demonstrate that tamoxifen is a first-line effective adjuvant therapy in women with a history of breast cancer and is an effective chemoprevention agent for women who are high risk for developing breast cancer.

Raloxifene is a member of the benzothiophene class of SERMs that has recently been approved for the prevention and treatment of osteoporosis. Raloxifene has not been evaluated for effectiveness as an adjuvant therapy for women with breast cancer. However, the Multiple Outcomes of Raloxifene (MORE) trial evaluated the effect of raloxifene on preventing breast cancer. The MORE trial was a randomized, placebo-controlled three-year study of 7705 postmenopausal women who have osteoporosis. In the MORE trial, 13 cases of breast cancer were found among the 5129 women in the raloxifene treatment group versus 27 among the 2576 women who received placebo (RR=0.24) after a median follow-up of 40 months. Like tamoxifen, raloxifene is effective at reducing the incidence of estrogen receptor positive tumors, but not estrogen receptor negative tumors. Additional evidence for a role of estrogens in promoting breast cancer comes from a recent study that showed raloxifene only prevents breast cancer in postmenopausal women that have detectable levels of serum estradiol.

Structure of Estrogens Receptors: The fact that SERMs only work on ER positive tumors indicates that they need to interact with estrogen receptors in order to exert its protective effects on the breast. There are two known estrogen receptors, ERα and ERβ, which are members of the steroid nuclear receptor superfamily. ERα was first cloned in 1986, and surprisingly about 10 years later a second ER was discovered, termed ERβ. ERα contains 595 amino acids, whereas ERβ contains 530 amino acids. Both receptors are modular proteins made up of three distinct domains. The amino-terminus domain (A/B domain) is the least conserved region, exhibiting only a 15% homology between ERα and ERβ. This domain harbors an activation function (AF-1) that can activate gene transcription in the absence of estradiol. The central region of ERs contains two zinc finger motifs that bind to an inverted palindromic repeat sequence separated by three nucleotides located in the promoter of target genes. The DNA binding domain (DBD) in ERα and ERβ are virtually identical, exhibiting 95% homology. The carboxy-terminus domain contains the ligand binding domain (LBD), which carries out several essential functions. The LBD contains a region that forms a large hydrophobic pocket, where estrogenic compounds bind, as well as regions involved in ER dimerization. The LBD also contains a second activation function (AF-2) that interacts with coregulatory proteins. AF-2 is required for both estrogen activation and repression of gene transcription. The LBDs of ERα and ERβ are only about 55% homologous. The striking differences in the amino acid composition of the ERα and ERβ LBDs may have evolved to create ERs that have distinct transcriptional roles. This would permit ERα and ERβ to regulate the activity of different genes and to elicit different physiological effects. This notion is supported by studies of ERα and ERβ knockout mice. For example, the ERα knockout mice have primitive mammary and uterine development, whereas the ERβ knockout mice develop normal mammary glands and uterus. These observations demonstrate that only ERα is required for the development of these tissues. Furthermore, we have found that ERα is more effective than ERβ at activating genes, whereas ERβ is more effective than ERα at repressing gene transcription.

### Mechanisms of Action of Estrogens

Estrogens can activate and/or repress gene transcription through a plurality of mechanisms. There are two characterized pathways for activation of gene transcription, the classical ERE (estrogen response element) pathway and the AP-1 pathway. There are at least three essential components necessary for estrogens to regulate the transcription of genes: the ERs (ERα and/or ERβ), the promoter element in target genes and coregulatory proteins. The binding of estradiol to the ER leads to a conformational change, which results in several key steps that initiate transcriptional pathways. First, the interaction of E₂ with ER leads to the dissociation of chaperone proteins from the ER; this exposes the ER's dimerization surface and DNA binding domain. Loss of the chaperone proteins allows the ERs to dimerize and bind to an ERE in the promoter region of a target gene.

Second, the binding of E₂ moves helix 12 of the ER's LBD to create a surface that assembles the AF-2 function of the ER. The AF-2 consists of a conserved hydrophobic pocket comprised of helices 3, 5 and 12 of the ER, which together form a binding surface for the p160 class of coactivator proteins (coactivators), such as steroid receptor coactivator-1 (SRC-1) or glucocorticoid receptor interacting protein 1 (GRIP 1). Coactivators (also known as “coregulators”) contain several repeat amino acid motifs comprised of LXXLL, which project into hydrophobic cleft surrounded by the AF-2's helices. The coactivators possess histone acetylase activity. It is thought that gene activation occurs after the ERs and coactivator proteins form a complex on the ERE that causes the acetylation of histone proteins bound to DNA. The acetylation of histones changes the chromatin structure so that the ER/coregulator complex can form a bridge between the ERE and basal transcriptional proteins that are assembled at the TATA box region of the target gene to initiate gene transcription.

Effect of SERMs on the ERE pathway: Unlike estrogens, SERMs do not activate the ERE pathway. Instead, the SERMs competitively block the effects of estrogens on the ERE pathway. Like estrogens, SERMs bind to ERα and ERβ with high affinity and cause the dissociation of chaperone proteins, ER dimerization and binding of ERs to the ERE. Thus, the antagonist action of SERMs occurs at a step distal to the binding of the ER to the promoter region. The molecular mechanism of the antagonist action of the SERMs has been clarified by the crystallization of the ERα and ERβ LBDs. It is clear from the structure of the ER LBDs that E₂, tamoxifen and raloxifene bind to the same binding pocket. However, tamoxifen and raloxifene contain a bulky side-chain that is absent in E₂. The ER x-ray structures have revealed that the bulky side chain of SERMs obstructs the movement of the LBD, which prevents the formation of a functional AF-2 surface. Remarkably, when a SERM binds to ERα a sequence (LXXML) in helix 12, which is similar to the LXXLL motif, interacts with the hydrophobic cleft of the AF-2 surface to occlude the coactivator recognition site. Thus, unlike estrogens, SERMs do not create a functional AF-2 surface; this prevents the binding of coactivators. Because the coactivator proteins do not bind to the AF-2 surface in the presence of SERMs, the activation pathway is abruptly halted. Instead of recruiting coactivator, ERs liganded with SERMs recruit corepressors, such as N-CoR.

These studies demonstrated that the antagonist properties of SERMs are due to at least three factors. First, SERMs bind to the same binding pocket as estrogens and competitively block their binding to the ERs. Second, SERMs prevent ER from interacting with coactivator proteins that are required for transcriptional activation of the ERE pathway. Third, SERMs recruit corepressors, which prevent transcriptional activation of genes. These actions of SERMs most likely explain how raloxifene and tamoxifen act as antagonists in breast cells to inhibit development of breast cancer.

SERMs are also more effective than E₂ at activating genes with an AP-1 element. In fact, E₂ is an antagonist of SERM-mediated activation of AP-1 elements. It has been postulated that SERMs exhibit agonistic actions in tissues, such as the bone and endometrium by activating the AP-1 pathway. Interestingly, SERMs are more potent at activating the AP-1 pathway in the presence of ERβ, which indicates that SERMs will trigger the AP-1 pathway more efficiently in tissues that are rich in ERβ. The role of the AP-1 pathway in estrogen-mediated breast carcinogenesis is unclear, because estrogens are much weaker at activating the AP-1 pathway compared to SERMs. However, it has been proposed that the AP-1 pathway may be involved in resistance to tamoxifen in breast tumors.

In accordance with aspects of the present invention, studies have been performed, which demonstrate that: ERβ is weaker than ERα at activating ERE-tkLuc; ERβ is more effective than ERα at repressing the TNF-RE-tkLuc; and that ERβ inhibits ERα-mediated transcriptional activation of ERE-tkLuc. Detailed experiments are discussed in the Examples section hereinafter.

The invention provides a composition that contains at least one isolated and purified compound of formula I or 6, as described herein. In particular, the composition contains at least one isolated and purified compound of formula 1, 2, 3 and/or 6. In some embodiments, the composition contains 2 or more, 3 or more or all 4 of compounds of formulae 1, 2, 3 and/or 6. In some embodiments, the composition contains 1 and 2; in some embodiments, the composition contains 1 and 3; in some embodiments, the composition contains 1 and 6; In some embodiments, the composition contains 1, 2 and 6; in some embodiments the composition contains 1, 3 and 6; in some embodiments, the composition contains 2 and 6; in some embodiments, the composition contains 3 and 6; in some embodiments, the composition contains 2, 3 and 6; in some embodiments, the composition contains 1, 2, 3 and 6. In some embodiments, the composition comprises 1 and 6. In some embodiments, the composition is an extract of Rheum palmatum rhizome, containing all three of 1-3 and 6. Also provided are methods of isolating compounds 1, 2, 3 and/or 6 from extracts of the rhizome of Rheum palmatum.

The species Rheum palmatum L of the Polygonaceae family is also variously referred to Rhubarb, Chinese rhubarb, East Indian rhubarb, sweet round-leaved dock, pieplant or da huang. Rheum palmatum L of the Polygonaceae family is a perennial shrub. It has very broad leaves and elongated, often reddish, petioles (leaf stalks). The aerial part is large, about 1.5-2 m tall and stout. The rhizomes and roots are stout; the stem is hollow, sulcate, subglabrous or muricate on the nodes. Petiole of basal leaf is terete, about as long as the blade and densely papilliferous; the leaf blade large, about as long as it is wide (40-60 cm) abaxially densely pubescent, adaxially sulcate to papilliferous, having 5 basal veins, base cordate, palmately divided into pinnatisect lobes, apex acuminate or narrowly acute. Stem leaves are smaller above. The ocrea are large, up to 15 cm, outside muricate. Panicle large; branches connivent, densely pubescent. Pedicel 2-2.5 mm, jointed below middle. The flowers small. Tepals 6, purple-red, rarely yellow-white, outer 3 elliptic to orbicular, smaller, 1-1.5 mm. Stamens not exceeding perianth. Ovary rhomboid-ovoid; style slightly deflexed; stigma inflated. Fruit oblong-ellipsoid to oblong, 8-9×7-7.5 mm, both ends retuse; wings ca. 2.5 mm wide, with longitudinal veins near margin.

Extract of Rheum palmatum

An extract of Rheum palmatum is first obtained by contacting plant parts comprising rhizome of Rheum palmatum with a suitable extraction medium for a suitable time and under suitable conditions to effect efficient extraction of the ERβ-selective estrogenic principles from the rhizome. The extraction medium is a suitable liquid solvent, e.g. aqueous lower alcohol, especially aqueous methanol, although aqueous ethanol (or a mixture of aqueous ethanol and methanol) is possible. The rhizome may be ground or otherwise comminuted to increase the contact surface area between the extraction solvent and the plant matter. Extraction may be carried out at room temperature or at an elevated temperature up to about 50° C. for a period from about 1 hour to about 72 hours. The plant matter may then be separated from the extraction medium to produce an extract containing, among other organic materials, a mixture of 1-3 and 6.

First Stage Separation p Alcohol (e.g. methanol) may the be removed in part or completely from the aqueous extraction medium to form an aqueous concentrate, which may then be charged to a solid phase extraction column or cartridge, e.g. a reverse-phase extraction cartridge. Partitioning of the extract may then be carried out by elution with serial aliquots of elution solvent, the serial aliquots ranging from polar to non-polar. In some embodiments, a suitable polar solvent is aqueous ammonium acetate (1-100 mM) and a suitable non-polar solvent is acetonitrile. Mixtures of these solvents may be prepared as elution solvents of intermediate polarity. In some embodiments, a suitable system for eluting active compounds from the solid phase comprises serial elution solvents as follows: (A) 100% aqueous ammonium acetate; (B) 75% aqueous ammonium acetate, 25% acetonitrile; (C) 50% aqueous ammonium acetate; 50% acetonitrile; (D) 25% aqueous ammonium acetate; 75% acetonitrile; (D) 100% acetonitrile. (All percentages (%) in vol/vol). Partitions having selective ERβ estrogenic activity may be saved and those lacking such activity may be discarded. In the foregoing aqueous ammonium acetate solutions, the ammonium acetate concentration may be in a suitable range, e.g. about 1 to about 100 mM, about 2 to about 50 mM, about 5 to about 20 mM or about 10 mM. It is also possible, especially in a scale-up environment, to run the elution solvent as a linear gradient from 100% aqueous ammonium acetate to 100% acetonitrile. Other suitable polar and non-polar solvents may also be substituted for those given herein under suitable conditions.

Final Stage Separation of Octahydroxyanthraquinone (6)

In the above-identified system, a fraction containing octahydroxyanthraquinone is obtained in fraction (C). This fraction may be loaded onto a Sephadex LH-20 substrate (e.g. in a cartridge or column) and eluted with methanol or other lower alcohol or mixture of lower alcohols. The eluted solvent containing octahydroxyanthraquinone may then be applied to a suitable reverse-phase separation substrate and partitioned with a suitable mobile phase. The separation substrate may be in or on a suitable support, such as a thin layer plate, a cartridge or a column of suitable capacity. A suitable mobile phase should be of suitable polarity for separating the desired product 6 from other materials on the substrate. In some embodiments, the substrate may be RP18 on a TLC plate, and the mobile phase may be a mixture of aqueous ammonium acetate and acetonitrile (e.g. a 6:4 mixture of 10 mM ammonium acetate: acetonitrile). In some such embodiments, five separate partitions are discemable, of which the second contains 6. The partition containing 6 may then be applied to a normal phase substrate, such as a silica gel substrate, on a suitable support (column, cartridge, TLC plate) and partitioned with a suitable solvent. In some embodiments, the stationary phase is a silica gel on a TLC plate and the developing solvent is hexane, ethyl acetate and trifluoroacetic acid in a suitable ratio (e.g. about 8:2:0.1 hexane:EtOAc:TFA). The partition containing 6 is obtained and is separated from the substrate by conventional methods. The identity of 6 may be confirmed by comparison against a known standard by one or a combination of methods, such as HPLC, ¹H NMR, ¹³C NMR, and/or mass spectrometry, as described in more detail in the example section, below.

### Isolation of Emodin (1), Aloe-Emodin (2) and Chrysophanol (3)

In the above-identified system, a mixture containing emodin (1), aloe-emodin (2) and chrysophanol (3) is eluted as fraction (D). This mixture may be applied to a normal phase separation substrate, such as silica, on a suitable support, such as a TLC plate, a cartridge or a column, and partitioned with a suitable mobile phase, such as a solvent having mixed polar and non-polar character. In some embodiments, the mixture is applied to a silica gel column and eluted with a solution of hexane, ethyl acetate and trifluoroacetic acid (e.g. an 8:2:0.1 mixture of hexane, EtOAc and TFA). Separate fractions containing chrysophanol (3), emodin (1) and aloe-emodin (2) may be obtained. Progress of the elution may be monitored, e.g. by reverse-phase TLC with a suitable mobile phase. In some embodiments, the mobile phase for TLC is a 1:1 mixture of ammonium acetate (10 mM) and acetonitrile. The identify of the separated compounds 1, 2 and 3 may be confirmed by comparison against a known standard by one or a combination of methods, such as HPLC, ¹H NMR, ¹³C NMR, and/or mass spectrometry, as described in more detail in the example section, below

Thus, the extract described above contains at a minimum one or more plant-derived compounds 1-3 and 6. (phytochemicals), optionally dissolved in a suitable solvent. A partially or completely evaporated extract may be reconstituted by adding a suitable diluent, e.g. ethyl acetate, water and/or ethanol, to form a reconstituted extract.

In some embodiments, compositions comprising plant extracts include pure extracts or partitioned extracts (including extracts in which one or more estrogenically active compounds in the extract have been enriched) and combinations of such extracts with one or more additional ingredients. In some embodiments, the compositions include those in a variety of physical forms, including solid, semi-solid, liquid, colloidal, etc. Where the compositions are intended for pharmaceutical use, the additional ingredients are pharmaceutically acceptable. Where the compositions according to the invention are intended for use in assays or other uses that are not directed toward a living body, the additional ingredient(s) may be either pharmaceutically acceptable or not.

In some embodiments, a pure extract may be combined with one or more organic solvents. Such organic solvents may be of various polarities. In some embodiments, suitable solvents include ethyl ethyl acetate, acetonitrile, hexanes, a (C₁-C₄) alcohol (e.g. methanol, ethanol, i-propanol, n-propanol, n-butanol, t-butanol, s-butanol, i-butanol, etc.), chloroform, acetone, cyclohexane, cycloheptane, petroleum ether, and other solvents, including those that are pharmaceutically acceptable and those that are generally regarded as safe (GRAS) for human consumption.

In some embodiments, the compositions comprise pure extracts or combinations of extracts with one or more additional solvents. In some embodiments, the extract includes a partitioned or further purified extract. Partitioning or purification may be conducted using various separation techniques, including chromatography. In some embodiments, the extract is a purified or partitioned extract obtained by means of anion exchange chromatography, cation exchange chromatography, reverse phase chromatography, normal phase chromatography, affinity chromatography or exclusion chromatography, to further concentrate active agents in the extract. In some embodiments, the purified or partitioned extract is obtained via one or more steps of liquid chromatography, such as high performance liquid chromatography (HPLC). In some embodiments, high performance liquid chromatography is preparative scale high performance liquid chromatography. In some embodiments, the HPLC is reverse phase or ion exchange chromatography. Other means of separation may also be used to purify or partition the extract, including separation in a separatory funnel or other bi- or multi-phasic separatory mechanism. In some embodiments, the purified or partitioned extract may be combined with one or more additional active or inactive ingredients, such as solvents, diluents, etc. In some embodiments, suitable solvents may include ethyl acetate, acetonitrile, hexanes, a (C₁-C₄) alcohol (e.g. methanol, ethanol, i-propanol, n-propanol, n-butanol, t-butanol, s-butanol, i-butanol, etc.), chloroform, acetone, cyclohexane, cycloheptane, petroleum ether, and other solvents, including those that are pharmaceutically acceptable and those that are generally regarded as safe (GRAS) for human consumption.

Suitable additional ingredients include solvents. Solvents may be subdivided into pharmaceutically acceptable and non-pharmaceutically acceptable solvents. In this context, it is to be understood that some pharmaceutically acceptable solvents include water for injection (WFI), which may be pH adjusted and/or buffered to a preselected pH or pH range, e.g. from about 2 to about 8, more specifically from about 4.0 to about 7.5, and more particularly from about 4.9 to about 7.2.

Pharmaceutically acceptable solvents may further comprise one or more pharmaceutically acceptable acids, bases, salts or other compounds, such as carriers, excipients, etc. Pharmaceutically acceptable acids include HCl, H₂SO₄ H₃PO₄, benzoic acid, etc. Pharmaceutically acceptable bases include NaOH, KOH, NaHCO₃, etc. Pharmaceutically acceptable salts include NaCl, NaBr, KCl, etc. Acids and bases may be added in appropriate proportions to buffer a pharmaceutically acceptable solution at a particular, pre-selected pH, especially a pH in the range of about 2-8, more especially in the range of about 5.0 to about 7.2

Plant extracts according to the present invention provide estrogenic activation of genes under control of the estrogen response element (ERE). Accordingly, in some cells an inventive plant extract possesses estrogenic properties—i.e. contacting a cell comprising an ERE and an ER (ERα, ERβ or both) with an inventive plant extract gives rise to stimulation of a gene under control of the ERE. In an in vitro cell system, ERE-mediated activation by an inventive estrogenic plant extract leads to expression of a gene that is operatively linked to the ERE. In particular embodiments, estrogenic interaction of an ER with an ERE linked to the minimal thymidine kinase promoter and the luciferase gene gives rise to enhanced luciferase expression. Thus, the plant extracts of the present invention may be used to identify ERα+ cell lines, ERα+ cell lines and/or ERα+/ERβ+ cell lines having an ERE-containing promoter operatively linked to a reporter gene, such as luciferase. Plant extracts of the present invention may also be used as assay reagents, including standards, for identifying compounds having estrogenic effects in ER+ cell lines.

In one such assay method, an inventive plant extract is first prepared at a known activity or concentration. Quantification of the inventive plant extract is conveniently carried out by taring a container, measuring into the container a known volume of the plant extract, reducing the plant extract by evaporation or lyophilization to produce a residue, and obtaining the mass of the container plus plant extract. The difference in mass between the container plus plant extract and the tare mass is the dry mass of the plant extract. The ratio of dry mass of plant extract per volume of plant extract is the concentration per unit volume. The plant extract may be used in its initial form, using the results of the foregoing quantitation method to specify its concentration. The residue can also be reconstituted by addition of water or another suitable solvent system to form a plant extract solution of known concentration.

Once the concentration of plant extract is known, a standard curve is prepared. In general the ER+ cells are contacted with the plant extract and a signal relating to estrogenic activity is recorded. In particular, an ER+ cell has a reporter gene under the control of an ERE. This ER+ cell is contacted with a plant extract of the invention, which gives rise to a reporter signal in proportion to the amount of plant extract added. This step may be carried out with multiple samples at the same plant extract concentration, at different plant extract concentrations, or both. As an example, nine samples may be tested: the first three at a first concentration, the next three at a concentration that is a half log greater than the first, and the next three at a concentration a whole log greater than first. The reporter signals are then observed and recorded, and the resulting data points (plant extract concentration versus reporter signal strength) are fitted to a standard curve by a conventional curve-fitting method (e.g. least squares).

To evaluate the estrogenic effect of a candidate compound, a candidate compound is contacted with E+ cells having the reporter gene under control of the ERE. The reporter gene signal is observed and compared to the standard curve to quantitate the candidate compound's relative estrogenic effect.

The ER+ cell line used in the foregoing method may be a cell line that naturally expresses ER, e.g. a human-derived ER+ breast cell carcinoma cell line. In some embodiments, the ER+ tissue is an immortalized human cell line, e.g. an immortalized bone marrow or breast cell line. Exemplary cell lines include human monocyte, osteoblast, malignant breast carcinoma and immortalized epithelial breast cell lines. Particular cell lines that may be mentioned include U937, U2OS, MDA-MB-435 and MCF-7 cell lines. Other ER+ cell lines, including immortalized cell lines, may also be used. Alternatively, the ER+ cell line may be a cell line that does not naturally express ER, such as a bacterial cell line, that has been transformed with an ER expression vector.

The ER+ cell line is transformed with a vector having a promoter containing an ERE that controls a reporter gene. For example, the vector may be a viral vector containing ERE, a minimal thymidine kinase promoter (tk) and a luciferase gene (Luc). An exemplary ERE-tk-Luk construct is depicted in SEQ ID NO:1, where the ERE is represented by nucleotides 1-, tk is represented by nucleotides nn-, and Luk is represented by nucleotides mm-. The construct is transfected into the target cell by known methods and expression of the ER-ERE-tk-Luk system is confirmed by e.g. performing the foregoing assay on putative ER+ cells in the presence of known quantities of E₂. Other methods of verifying successful transformation of ER+ cells include immunostaining with known ER antibodies.

The ERE-containing promoter is a DNA containing an ERE sequence and a promoter sequence. The promoter sequence is an art-recognized promoter sequence, such as the minimal thymidine kinase (tk) promoter sequence. (See SEQ ID NO:1, nucleotides nn-). Other ERE-containing promoters are possible and are within the scope of the instant invention. The ERE and promoter sequence operate together to control expression of the reporter gene. As described herein, the estrogenic compound (plant extract or E₂, for example) binds to the ER, giving rise to ER dimer and forming the AF-2 surface. The ER dimer then binds to the ERE, activating the gene under control of the promoter. In some embodiments, the ERE is directly upstream of (5′- to) the promoter, to which it is directly ligated. As an example, the ERE-tk promoter construct is shown in SEQ ID NO: 1, nucleotides 1-nn-1.

The reporter gene is a gene which, when expressed, gives rise to a detectable signal. The luciferase gene is a suitable reporter gene because it gives rise to the protein luciferase, which generates a detectable light signal in the presence of a single reagent, luciferin. In particular, the cDNA of the luciferase gene is expressed to produce the 62 kDa enzymatic protein, luciferase. The luciferase enzyme catalyzes the reaction of luciferin and ATP in the presence of Mg²⁺ and oxygen to form oxyluciferin, AMP, pyrophosphate (PPi) and emitted light. The emitted light is yellow-green (560 nm), and may easily be detected using a standard photometer. Because ATP, O₂ and Mg²⁺ are already present in cells, this reporter gene only requires addition of the reagent luciferin to produce a detectable signal, and is especially well-suited for use in assays of the present invention. Other reporter genes that may be mentioned as being available in the art include chloramphenicol transacetylase (CAT), neomycin phosphotransferase (neo) and beta-glucuronidase (GUS).

In some assay methods of the invention, it is useful to further characterize the standard plant extract by comparison with one or more estrogenic compounds, SERMs, etc. Such assay methods are performed essentially as described above, making the proper substitutions of standard estrogenic compound and/or SERMs for plant extract in the appropriate parts of the method.

Plant extracts according to the present invention also repress gene expression by the TNF RE-mediated pathway. In some cases, plant extracts of the invention repress gene expression in vitro, especially in cells having a reporter gene (e.g. the luciferase gene, Luc) under control of a TNF RE. In some cases, plant extracts of the invention repress expression of TNF-α, which is a cytokine produced primarily by monocytes and macrophages. This cytokine is found in synovial cells and macrophages in various tissues, and has been strongly implicated in rheumatoid arthritis (RA). TNF-α is also expressed in other inflammatory diseases, and also as a response to endotoxins from bacteria. As repressors of TNF expression via the TNF RE repressed pathway, plant extracts of the invention are of interest in the treatment of inflammatory disorders associated with elevated levels of TNF.

In some embodiments of the invention, a cell line is prepared, which expresses one or both of ERα and ERβ as well as a reporter gene under control of TNF RE. The TNF RE is generally upstream of (5′- to) the reporter gene, and signal detection is carried out as previously described herein. The sequence of DNA having a reporter gene, in this case luciferase gene, under control of TNF RE is set forth in SEQ ED NO:2. Nucleotides 1-correspond to the TNF RE, while nucleotides nn- corresponds to the luciferase gene.

The foregoing cell TNF RE-containing cell system further contains one or more copies of an ER gene—i.e. ERα, ERβ or both. The ER+ cell line used in the foregoing method may be a cell line that naturally expresses ER, e.g. a human-derived ER+ breast cell carcinoma cell line. In some embodiments, the ER+ tissue is an immortalized human cell line, e.g. an immortalized bone marrow or breast cell line. Exemplary cell lines include human monocyte, osteoblast, malignant breast carcinoma and immortalized epithelial breast cell lines. Particular cell lines that may be mentioned include U937, U2OS, MDA-MB-435 and MCF-7 cell lines. Other ER+ cell lines, including immortalized cell lines, may also be used. Alternatively, the ER+ cell line may be a cell line that does not naturally express ER, such as a bacterial cell line, that has been transformed with an ER expression vector.

In the presence of a predetermined amount of luciferin, and in the absence of an estrogenic compound, e.g. E₂ or a plant extract of the invention, the cell system emits a yellow light (560 nm) at an intensity, called the “control intensity” or the “baseline intensity”. Light emission at 560 nm is conveniently quantified in optical density units (O.D._{560 nm}). Upon addition of an estrogenic compound, e.g. E₂ or one of the inventive plant extracts, the intensity of 560 nm light emissions is attenuated as compared to the control. Remarkably, in the presence of a SERM, such as tamoxifen or raloxifene, luciferase expression increases and 560 nm light emission intensity also increases. Thus, plant extracts of the invention are capable of inducing an estrogenic TNF RE-controlled repression of gene expression.

The TNF RE-containing cell system can be used in an assay method according to the invention. In the inventive assay methods, the attenuation of luciferase activity (i.e. decreased emission of 560 nm light), correlates with increased estrogenic activity, whereas activation of luciferase activity (i.e. increased emission at 560 nm), correlates with anti-estrogenic activity. Standard curves may be prepared using known quantities of the inventive plant extracts, as described herein. Such standard curves may be further augmented by using other known estrogenic or anti-estrogenic standards, such as E₂ or some other known estrogenic compound, and/or an anti-estrogenic SERM such as tamoxifen or raloxifene.

Cells from the transformed E+ cell line are then exposed to a candidate compound, the luciferase signal observed, and the signal compared to the previously prepared standard curve(s), as described herein. A compound that causes an increase of luciferase activity as compared to control (baseline), will be characterized as an anti-estrogenic SERM, whereas a compound that causes a decrease in luciferase activity versus control will be classified as estrogenic. The estrogenic or anti-estrogenic effect can then be quantified by comparing the degree of luciferase expression decrease or increase against the decrease brought about by the inventive plant extract, and optionally the respective signal decrease or increase brought about by E₂, tamoxifen and/or raloxifene.

Plant extract compositions of the present invention also antagonize the interaction of E₂-ER with ERE. In particular, it has been shown in that extracts of Rheum palmatum L of the Polygonaceae family antagonize the activation of ERE-tk-Luc by E₂ by directly interacting with ERβ and ERα. As antagonists of E₂-ER activation of ERE-controlled genes, the inventive plant extract compositions are considered to be similar in effect to tamoxifen, possessing prophylactic, palliative and/or anti-proliferative activity against breast cancer and uterine cancer.

The invention provides in vivo estrogenic methods of using the inventive compositions. In general, in vivo methods comprise administering to a subject an amount of the plant extract sufficient to bring about an estrogenic effect in the subject. The in vivo methods will give rise to estrogenic ERE-controlled gene activation, TNF RE-controlled gene repression (e.g. TNF-α repression), or both. Thus, the in vivo methods will give rise to varied positive phenotypic effects in vivo.

The subject may be a mammal, such as a mouse, rat, rabbit, monkey, chimpanzee, dog, cat or a sheep, and is generally female. The subject may also be human, especially a human female. In some embodiments, the subject is a post-menopausal or post-oophorectomic female, and is in need of estrogenic therapy. In such case, the subject may be suffering from climacteric symptoms, such as hot flashes, insomnia, vaginal dryness, decreased libido, urinary incontinence and depression. In other such cases, the subject may be susceptible to, or suffering from, osteoporosis. Suitable in vivo methods include treatment and/or prevention of medical indications that are responsive to estrogen replacement therapy.

Administration of the compositions according to the present invention will be via a commonly used administrative route so long as one or more of the plant extracts is available to target tissue via that route. Some administrative routes that may be mentioned include: oral, nasal, buccal, rectal, vaginal and/or topical (dermal). Alternatively, administration may be by orthotopic, intradermal, subcutaneous, intramuscular, intraperitoneal or intravenous injection. Such compositions would normally be administered as pharmaceutically acceptable compositions, described supra.

Treatment (and its grammatical variants—e.g. treat, to treat, treating, treated, etc.) of a disease, disorder, syndrome, condition or symptom includes those steps that a clinician would take to identify a subject to receive such treatment and to administer a composition of the invention to the subject. Treatment thus includes diagnosis of a disease, syndrome, condition or symptom that is likely to be ameliorated, palliated, improved, eliminated, cured by administering the estrogenic plant extract of the invention to the subject. Treatment also includes the concomitant amelioration, palliation, improvement, elimination, or cure of the disease, disorder, syndrome, condition or symptom. In some embodiments, treatment implies prevention or delay of onset of a disease, disorder, syndrome, condition or symptom (i.e. prophylaxis), prevention or delay of progression of a disease, disorder, syndrome, condition or symptom, and/or reduction in severity of a disease, disorder, syndrome, condition or symptom. In the case of neoplastic growth in particular, treatment includes palliation, as well as the reversal, halting or delaying of neoplastic growth. In this regard, treatment also includes remission, including complete and partial remission. In the case of climacteric symptoms, treatment includes prevention and palliation of various symptoms.

Prevention (and its grammatical variants) of a disease, disorder, syndrome, condition or symptom includes identifying a subject at risk to develop the disease, disorder, syndrome, condition or symptom, and administering to that subject an amount of the inventive plant extract sufficient to be likely to obviate or delay the onset of said disease, disorder, syndrome, condition or symptom. In some cases, prevention includes identifying a post-menopausal woman who the clinician believes, applying a competent standard of medical care, to be in need of hormone replacement therapy, and administering a plant extract of the present invention to the woman, whereby one or more climacteric symptoms is blocked or delayed. In some embodiments, prevention of osteoporosis includes identifying a post-menopausal woman who the clinician believes, applying a competent standard of medical care, to be at risk for developing osteoporosis, and administering a plant extract of the present invention to the woman, whereby the onset of bone loss is blocked or delayed.

Palliation includes reduction in the severity, number and/or frequency of occurrences of an a disease, disorder, syndrome, condition or symptom. Palliation of climacteric symptoms includes reducing the frequency and/or severity of hot flashes, insomnia, incontinence, depression, etc.

Treatment of osteoporosis includes identifying a person, such as a post-menopausal woman, at risk for bone loss, and administering a plant extract of the present invention to the woman, whereby bone loss is reduced in severity, delayed in onset, or prevented. In some embodiments, treatment of osteoporosis can also include addition of bone mass.

The invention further provides methods of making the inventive extracts of Rheum palmatum L of the Polygonaceae family. The invention specifically provides a method of making an inventive estrogenic plant extract. The method includes obtaining a quantity of plant matter from a plant of the species Rheum palmatum L of the Polygonaceae family, optionally comminuting the plant matter, contacting said plant matter with an extraction medium, and separating the plant matter from the extraction medium.

In some embodiments, the plant species are of the plant species Rheum palmatum L of the Polygonaceae family are various cultivars of Rheum palmatum L of the Polygonaceae family

Plant matter means any part or parts of at least one plant from the species Rheum palmatum L of the Polygonaceae family. Plant matter includes the whole plant or any part or parts of the plant, such as the root, bark, wood, leaves, flowers (or flower such as: sepals, petals, stamens, pistils, etc.), fruit, seeds and/or parts or mixtures of any of the foregoing. Plant matter may be fresh cut, dried (including freeze dried), frozen, etc. Plant matter may also be whole or separated into smaller parts. For example, leaves may be chopped, shredded or ground; roots may be chopped or ground; fruit may be chopped, sliced or blended; seeds may be chopped or ground; stems may be shredded, chopped or ground. In particular embodiments of the invention, the plant parts used are the leaves of Rheum palmatum L of the Polygonaceae family

Plant extract compositions of the invention contain at least one extract of an Rheum palmatum L of the Polygonaceae family. An “extract” is a solution, concentrate or residue that results when a plant part is contacted with an extraction solvent under conditions suitable for one or more compounds from the plant to partition from the plant matter into the extraction solvent; the solution is then optionally reduced to form a concentrate or a residue.

Suitable extraction media for the present invention include water and ethyl alcohol. Specifically, where water is the extraction solvent, purified water is suitable. Purified water includes distilled water, deionized water, water for injection, ultrafiltered water, and other forms purified of water. Ethyl alcohol that is employed in some embodiments of the invention is grain ethanol, and in particular undenatured ethanol (e.g. pure grain ethanol, optionally containing some water, e.g. up to about 10% water). In some embodiments, the extraction solvent is water, ethanol, or a mixture thereof. A concentrate or residue may be prepared by reducing (e.g. evaporating or lyophilizing) the extraction solution. Whether in the original extraction solvent, reduced concentrate, or residue form, each of these preparations is considered an “extract” for the purposes of the invention.

A method of producing the plant extract according to the invention optionally comprises first comminuting the plant matter in order to increase its surface area to volume ratio and to concomitantly increase efficiency of the extraction process. Methods of comminuting plant matter include grinding, chopping, blending, shredding, pulverizing, triturating, etc.

The extraction medium (solvent) is then contacted with the plant matter under conditions suitable for causing one or more phytochemicals, in particular estrogenic phytochemicals, to partition from the plant matter into the extraction medium. Such conditions include, in some cases, heating the extraction medium to a temperature above room temperature, agitation, contact time, etc. Exemplary temperatures for extraction are from about 50° C. to the boiling point of the extraction solvent. Where water is the extraction solvent, the extraction temperature is generally from room temperature to about 100° C.; temperatures of from about 50° C. to about 80° C. are especially suitable, and temperatures of about 75° C. are particularly suitable. In the case of ethanol as an extraction solvent, the extraction temperature is generally from about room temperature to about 78.5° C.; temperatures of from about 50° C. to about 78° C. are especially suitable and a temperature of about 75° C. is particularly suitable. The person of skill in the art will recognize that the proper balance should be drawn between extraction efficiency on the one hand and phytochemical compound stability on the other.

Once the extraction medium and the plant matter are combined, they are optionally agitated to ensure efficient exchange of estrogenic compound from the plant matter into the extraction medium, and are left in contact for a time sufficient to extract a useful amount of phytochemical compound from the plant matter into the extraction medium. After such time has elapsed (e.g. from about 5 min. to about 10 hr., more particularly from about 10 min. to about 5 hr., especially about 30 min. to about 2 hr.), the extraction medium containing the phytochemical compounds is separated from the plant matter. Such separation is accomplished by an art-recognized method, e.g. by filtration, decanting, etc.

A composition according to the invention includes an inventive plant extract or a composition comprising an inventive plant extract of the invention. In such embodiments, the inventive composition will optionally contain one or more additional ingredients. Such additional ingredients may be inert or active. Inert ingredients include solvents, excipients and other carriers. Active ingredients include active pharmaceutical ingredients (APIs), including those that exhibit synergistic activity in combination with the inventive plant extract.

Extracts of Rheum palmatum

In some embodiments, the plant species are of the plant species Rheum palmatum are various cultivars of Rheum palmatum.

Plant matter means any part or parts of at least one plant from the species Rheum palmatum, especially the spines (thorns) thereof In general, plant matter may include other parts of the whole plant or any part or parts of the plant, such as the root, bark, wood, leaves, flowers (or flower such as: sepals, petals, stamens, pistils, etc.), fruit, seeds and/or parts or mixtures of any of the foregoing, although in the currently preferred embodiments, the plant parts used for preparation of the extracts and pharmaceutical compositions comprising said extracts described herein are the spines (a.k.a. thorns) of Rheum palmatum. Plant matter may be fresh cut, dried (including freeze dried), frozen, etc. Plant matter may also be whole or separated into smaller parts. For example, leaves may be chopped, shredded or ground; roots may be chopped or ground; fruit may be chopped, sliced or blended; seeds may be chopped or ground; stems may be shredded, chopped or ground. In particular embodiments of the invention, the plant parts used are the spines (thorns) of Rheum palmatum.

Plant extract compositions of the invention contain at least one extract of an Rheum palmatum. An “extract” is a solution, concentrate or residue that results when a plant part is contacted with an extraction solvent under conditions suitable for one or more compounds from the plant to partition from the plant matter into the extraction solvent; the solution is then optionally reduced to form a concentrate or a residue.

Suitable extraction media for the present invention include water and ethyl alcohol. Specifically, where water is the extraction solvent, purified water is suitable. Purified water includes distilled water, deionized water, water for injection, ultrafiltered water, and other forms purified of water. Ethyl alcohol that is employed in some embodiments of the invention is grain ethanol, and in particular undenatured ethanol (e.g. pure grain ethanol, optionally containing some water, e.g. up to about 10% water). In some embodiments, the extraction solvent is water, ethanol, or a mixture thereof. A concentrate or residue may be prepared by reducing (e.g. evaporating or lyophilizing) the extraction solution. Whether in the original extraction solvent, reduced concentrate, or residue form, each of these preparations is considered an “extract” for the purposes of the invention.

A method of producing the plant extract according to the invention optionally comprises first comminuting the plant matter in order to increase its surface area to volume ratio and to concomitantly increase efficiency of the extraction process. Methods of comminuting plant matter include grinding, chopping, blending, shredding, pulverizing, triturating, etc.

The extraction medium (solvent) is then contacted with the plant matter under conditions suitable for causing one or more phytochemicals, in particular selective estrogenic phytochemicals, to partition from the plant matter into the extraction medium. Such conditions include, in some cases, heating the extraction medium to a temperature above room temperature, agitation, contact time, etc. Exemplary temperatures for extraction are from about 50° C. to the boiling point of the extraction solvent. Where water is the extraction solvent, the extraction temperature is generally from room temperature to about 100° C.; temperatures of from about 50° C. to about 80° C. are especially suitable, and temperatures of about 75° C. are particularly suitable. In the case of ethanol as an extraction solvent, the extraction temperature is generally from about room temperature to about 78.5° C.; temperatures of from about 50° C. to about 78° C. are especially suitable and a temperature of about 75° C. is particularly suitable. The person of skill in the art will recognize that the proper balance should be drawn between extraction efficiency on the one hand and phytochemical compound stability on the other.

Once the extraction medium and the plant matter are combined, they are optionally agitated to ensure efficient exchange of selective estrogenic compound from the plant matter into the extraction medium, and are left in contact for a time sufficient to extract a useful amount of phytochemical compound from the plant matter into the extraction medium. After such time has elapsed (e.g. from about 5 min. to about 10 hr., more particularly from about 10 min. to about 5 hr., especially about 30 min. to about 2 hr.), the extraction medium containing the phytochemical compounds is separated from the plant matter. Such separation is accomplished by an art-recognized method, e.g. by filtration, decanting, etc.

A composition according to the invention includes an herein-described plant extract or a composition comprising an herein-described plant extract of the invention. In such embodiments, the herein-described composition will optionally contain one or more additional ingredients. Such additional ingredients may be inert or active. Inert ingredients include solvents, excipients and other carriers. Active ingredients include active pharmaceutical ingredients (APIs), including those that exhibit synergistic activity in combination with the herein-described plant extract.

Some embodiments disclosed herein provide a pharmaceutical compositions comprising an extract of the taxonomic species Rheum palmatum. An “extract” is a composition of matter prepared in part by contacting an extraction medium (solvent) with plant matter under conditions suitable for drawing one or more chemical compounds from the plant matter into the extraction medium, forming an extraction solution. The extraction solution is then separated from the plant matter, and is optionally diluted or concentrated (e.g. by evaporation, sublimation or lyophilization) to form the extract.

The species Rheum palmatum is a deciduous tree growing to 12 m at a medium rate. The flowers are hermaphroditic, have both male and female organs, and are pollinated by insects. It can fix Nitrogen. The plant prefers light (sandy), medium (loamy) and heavy (clay) soils and requires well-drained soil. The plant prefers acid, neutral and basic (alkaline) soils. It cannot grow in the shade. It requires dry or moist soil and can tolerate drought. It can tolerate atmospheric pollution. Trees have a light canopy, they come into leaf late in the spring and drop their leaves in early autumn.

In particular embodiments, spines are harvested from the tree and contacted with the extraction medium within a short period after harvesting. The extraction medium is a suitable liquid solvent, e.g. ethyl acetate, water or ethanol. The extraction medium is in some cases ethyl acetate, water, ethanol or another relatively polar liquid solvent. In some cases, the extraction medium is either diluted or reduced. The extraction medium may be fully reduced, whereby the extract takes the form of a residue (residual extract). Thus, the extract contains at a minimum one or more plant-derived compounds (phytochemicals), optionally dissolved in a solvent, which are drawn into the extraction medium through one or more steps of contacting the extraction medium and the plant or plant parts. A concentrated or residual extract may be reconstituted by adding a suitable diluent, e.g. ethyl acetate, water and/or ethanol, to form a reconstituted extract.

In some embodiments, compositions comprising plant extracts include pure extracts or partitioned extracts (including extracts in which one or more selective estrogenic active compounds in the extract have been enriched) and combinations of such extracts with one or more additional ingredients. In some embodiments, the compositions include those in a variety of physical forms, including solid, semi-solid, liquid, colloidal, etc. Where the compositions are intended for pharmaceutical use, the additional ingredients are pharmaceutically acceptable. Where the compositions according to the invention are intended for use in assays or other uses that are not directed toward a living body, the additional ingredient(s) may be either pharmaceutically acceptable or not.

In some embodiments, a pure extract may be combined with one or more organic solvents. Such organic solvents may be of various polarities. In some embodiments, suitable solvents include ethyl acetate, acetonitrile, hexanes, a (C₁-C₄) alcohol (e.g. methanol, ethanol, i-propanol, n-propanol, n-butanol, t-butanol, s-butanol, i-butanol, etc.), chloroform, acetone, cyclohexane, cycloheptane, petroleum ether, and other solvents, including those that are pharmaceutically acceptable and those that are generally regarded as safe (GRAS) for human consumption.

In some embodiments, the compositions comprise pure extracts or combinations of extracts with one or more additional solvents. In some embodiments, the extract includes a partitioned or further purified extract. Partitioning or purification may be conducted using various separation techniques, including chromatography. In some embodiments, the extract is a purified or partitioned extract obtained by means of anion exchange chromatography, cation exchange chromatography, reverse phase chromatography, normal phase chromatography, affinity chromatography or exclusion chromatography, to further concentrate active agents in the extract. In some embodiments, the purified or partitioned extract is obtained via one or more steps of liquid chromatography, such as high performance liquid chromatography (HPLC). In some embodiments, high performance liquid chromatography is preparative scale high performance liquid chromatography. In some embodiments, the HPLC is reverse phase or ion exchange chromatography. Other means of separation may also be used to purify or partition the extract, including separation in a separatory funnel or other bi- or multi-phasic separatory mechanism. In some embodiments, the purified or partitioned extract may be combined with one or more additional active or inactive ingredients, such as solvents, diluents, etc. In some embodiments, suitable solvents may include ethyl acetate, acetonitrile, hexanes, a (C₁-C₄) alcohol (e.g. methanol, ethanol, i-propanol, n-propanol, n-butanol, t-butanol, s-butanol, i-butanol, etc.), chloroform, acetone, cyclohexane, cycloheptane, petroleum ether, and other solvents, including those that are pharmaceutically acceptable and those that are generally regarded as safe (GRAS) for human consumption.

Suitable additional ingredients include solvents. Solvents may be subdivided into pharmaceutically acceptable and non-pharmaceutically acceptable solvents. In this context, it is to be understood that some pharmaceutically acceptable solvents include water for injection (WFI), which may be pH adjusted and/or buffered to a preselected pH or pH range, e.g. from about 2 to about 8, more specifically from about 4.0 to about 7.5, and more particularly from about 4.9 to about 7.2.

Pharmaceutically acceptable solvents may further comprise one or more pharmaceutically acceptable acids, bases, salts or other compounds, such as carriers, excipients, etc. Pharmaceutically acceptable acids include HCl, H₂SO₄ H₃PO₄, benzoic acid, etc. Pharmaceutically acceptable bases include NaOH, KOH, NaHCO₃, etc. Pharmaceutically acceptable salts include NaCl, NaBr, KCl, etc. Acids and bases may be added in appropriate proportions to buffer a pharmaceutically acceptable solution at a particular, pre-selected pH, especially a pH in the range of about 2-8, more especially in the range of about 5.0 to about 7.2.

Pharmaceutical Compositions

Extracts of Rheum palmatum may be prepared as above in either solution or dried form. In a solution form, an extract of Rheum palmatum may be administered in the form a flavored or unflavored tea. In some embodiments some flavoring, e.g. sweetening, may be desirable to counteract the bitter flavor of the extract. Solutions can also be prepared from dried extract, in tea or elixir forms. Again, flavoring, such as sweetening may be desirable. Taste-masking may be employed to improve patient acceptance of the pharmaceutical composition.

A dried extract may be formulated as an orally-available form, such as in a capsule, tablet, caplet, etc. A capsule may be prepared by measuring a suitable amount of the dry extract into one or more gelatin capsule shells and assembling the capsule(s). Tablets and caplets may be prepared by combining the dry extract with one or more binders and optionally one or more disintegrants. Tablets, caplets, capsules, etc. may be coated, e.g. with an enteric coating, to prevent stomach upset.

Either a dried extract or a concentrated extract solution may be combined with one or more gelling agents and inserted into a gel capsule. Alternatively, a dried extract or concentrated extract solution may be combined with a gelling agent and optionally one or more flavoring agents for oral administration as an edible gel or a non-flavored variant may be administered as a rectal suppository gel or gel capsule.

A unit dose of extract is characterized by an equivalent amount of dried extract contained within the dosage form. For example, in some embodiments, a unit dosage may contain 1 mg to about 10 g of dried extract, or the equivalent thereof. In some embodiments, the unit dose will contain about 1 mg to about 10 mg, about 1 mg to about 100 mg, about 1 mg to about 1000 mg (1 g), about 1 mg to about 10000 mg (10 g) of dried extract, or the equivalent thereof. In some embodiments, the unit dose contains about 10 mg to about 100 mg, about 10 mg to about 1000 mg or about 10 mg to about 10000 mg of dried extract or the equivalent thereof. In some embodiments, the unit dose contains about 100 mg to about 5000, about 100 mg to about 2500 mg, about 100 mg to about 2000 mg, about 100 mg to about 1500 mg, about 100 to about 1000, about 100 to about 800 mg of dried extract, or the equivalent thereof. An equivalent of a dried extract of Rheum palmatum is an amount of a dry, liquid, gel or other mixture of Rheum palmatum containing the same amount of selective estrogenic actiye as a dried extract of Rheum palmatum. Thus, 30 mL of a tea containing 0.090 mg/mL of dried extract of Rheum palmatum is a unit dose equivalent to 15 mg of dried Rheum palmatum; and a tablet containing 100 mg each of dried extract of Rheum palmatum, a binder, a filler, a disintegrant is equivalent to 100 mg of dried extract neat.

### Methods of Treatment

The compositions comprising extracts of Rheum palmatum as described herein possess selective Rheum palmatum have selective estrogenic actively in estrogen receptor negative (ER-negative) cancer cells, such as ER-negative breast cancer and prostate cancer cells. Hence, it is expected that they will have activity in the treatment of various disease states that are characterized by hyperproliferation of cells, such as those caused by failure of normal selective estrogenic processes in an organism, organ, tissue or cell line. Among the disease states envisioned as being treatable with the compositions described herein is cancer, including, but not limited to bone cancer, brain stem glioma, breast cancer, cancer of the adrenal gland, cancer of the anal region, cancer of the bladder, cancer of the endocrine system, cancer of the esophagus, cancer of the head or neck, cancer of the kidney or ureter, cancer of the parathyroid gland, cancer of the penis, cancer of the small intestine, cancer of the thyroid gland, cancer of the urethra, carcinoma of the cervix, carcinoma of the endometrium, carcinoma of the fallopian tubes, carcinoma of the renal pelvis, carcinoma of the vagina, carcinoma of the vulva, chronic or acute leukemia, colon cancer, cutaneous or intraocular melanoma, glioma, Hodgkin's Disease, lung cancer, lymphocytic lymphomas, neoplasms of the central nervous system (CNS), ovarian cancer, pancreatic cancer, pituitary adenoma, primary CNS lymphoma, prostate cancer, rectal cancer, renal cell carcinoma, a sarcoma, e.g. of soft tissue, skin cancer, spinal axis tumors, stomach cancer or uterine cancer. In some embodiments the composition described herein is administered to a patient who has been diagnosed with one or more cancers selected from among the solid tumors, such as breast, lung, colon, brain, prostate, stomach, pancreatic, ovarian, skin (melanoma), endocrine, uterine, testicular and bladder cancer.

In some embodiments, compositions comprising extracts of Rheum palmatum described herein are effective to treat a benign proliferative disease, such as benign prostatic hypertrophy, psoriasis or restenosis (e.g. of an implanted stent).

In some embodiments, one or more compositions comprising extracts of Rheum palmatum described herein may be combined with another agent that is useful for the treatment of abnormal cell growth, such as cancer, solid tumors, benign hyperproliferative disease, etc. Such additional agent may be selected from among the mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxic agents, anti-hormones, and anti-androgens.

An effective dose of a composition comprising an extract of Rheum palmatum is an amount effective to produce a therapeutic effect in a multicellular organism as described herein. In some embodiments, the effective dose is an amount sufficient to induce apoptosis in one or more populations of hyperproliferative cells in the organism. In some embodiments, the effective dose is an amount sufficient to cause relief of one or more symptoms of hyperproliferative cellular disease, such as cancer, in the organism. In some embodiments, the effective dose is an amount sufficient to significantly slow the progression of hyperproliferative cellular disease, to cause partial or complete remission of said hyperproliferative cellular disease, to provide partial or complete prophylaxis against recurrence, spread or malignant growth of said hyperproliferative cellular disease. In some embodiments the dose may be critical to the success of the therapeutic regime. As the extracts of Rheum palmatum are deemed to be largely non-toxic, the effective dose may be varied from about 1 mg to about 10 g per patient per day of dried extract, or the equivalent thereof in a solution or other pharmaceutically acceptable form, as discussed in more detail below. In some embodiments, the effective dose is about 1 mg to about 10 mg, about 1 mg to about 100 mg, about 1 mg to about 1000 mg (1 g), about 1 mg to about 10000 mg (10 g) per patient per day. In some embodiments, the effective dose is about 10 mg to about 100 mg, about 10 mg to about 1000 mg or about 10 mg to about 10000 mg per patient per day. In some embodiments, the effective dose is about 100 mg to about 5000, about 100 mg to about 2500 mg, about 100 mg to about 2000 mg, about 100 mg to about 1500 mg, about 100 to about 1000, about 100 to about 800 mg per patient per day. In some embodiments, treatment days may be altered with non-treatment days. For example, treatment may be commenced on day 1 with an effective dose as described above, with administration of the effective dose repeated on days 3, 5, 7 (or 8), 9, 11, 13, etc. Treatment may be administered once a day for a full week, followed by a week off treatment, followed by at least one additional week on treatment. Treatment with the extract of Rheum palmatum may also be alternated with another anti-cancer treatment, or may be combined with another anti-cancer treatment to take advantage of the combined effects of the cancer treatments.

Additional cancer treatments can include, but are not limited to, surgical excision of all or part of a solid tumor, radiation treatment, adjunctive chemotherapy, anti-inflammatory drugs, analgesic drugs, etc.

Treatment (and its grammatical variants—e.g. treat, to treat, treating, treated, etc.) of a disease, disorder, syndrome, condition or symptom includes those steps that a clinician would take to identify a subject to receive such treatment and to administer a composition of the invention to the subject. Treatment thus includes diagnosis of a disease, syndrome, condition or symptom that is likely to be ameliorated, palliated, improved, eliminated, cured by administering the selective estrogenic plant extract of the invention to the subject. Treatment also includes the concomitant amelioration, palliation, improvement, elimination, or cure of the disease, disorder, syndrome, condition or symptom. In some embodiments, treatment implies prevention or delay of onset of a disease, disorder, syndrome, condition or symptom (i.e. prophylaxis), prevention or delay of progression of a disease, disorder, syndrome, condition or symptom, and/or reduction in severity of a disease, disorder, syndrome, condition or symptom. In the case of neoplastic growth in particular, treatment includes palliation, as well as the reversal, halting or delaying of neoplastic growth. In this regard, treatment also includes remission, including complete and partial remission. In the case of climacteric symptoms, treatment includes prevention and palliation of various symptoms.

Prevention (and its grammatical variants) of a disease, disorder, syndrome, condition or symptom includes identifying a subject at risk to develop the disease, disorder, syndrome, condition or symptom, and administering to that subject an amount of the herein-described plant extract sufficient to be likely to obviate or delay the onset of said disease, disorder, syndrome, condition or symptom. In some cases, prevention includes identifying a post-menopausal woman who the clinician believes, applying a competent standard of medical care, to be in need of hormone replacement therapy, and administering a plant extract of the present invention to the woman, whereby one or more climacteric symptoms is blocked or delayed. In some embodiments, prevention of osteoporosis includes identifying a post-menopausal woman who the clinician believes, applying a competent standard of medical care, to be at risk for developing osteoporosis, and administering a plant extract of the present invention to the woman, whereby the onset of bone loss is blocked or delayed.

Palliation includes reduction in the severity, number and/or frequency of occurrences of an a disease, disorder, syndrome, condition or symptom. Palliation of climacteric symptoms includes reducing the frequency and/or severity of hot flashes, insomnia, incontinence, depression, etc.

Administration of Extracts of Rheum palmatum

Administration of the compositions according to the present invention will be via a commonly used administrative route so long as one or more of the plant extracts is available to target tissue via that route. Some administrative routes that may be mentioned include: oral, nasal, buccal, rectal, vaginal and/or topical (dermal). Alternatively, administration may be by orthotopic, intradermal, subcutaneous, intramuscular, intraperitoneal or intravenous injection. Such compositions would normally be administered as pharmaceutically acceptable compositions, described supra.

### Examples

The invention may be more fully appreciated with reference to the following illustrative and non-limiting examples.

### Example 1

### Isolation and in vitro Testing of Compounds Isolated from Rheum palmatum

Rhizomes of Rheum palmatum (Polygonaceae) were subjected to activity-guided isolation where a series of anthraquinones, 1-3, and 6, were isolated. The isolated and two purchased (4, 5) anthraquinones, were tested for their ERα and ERβ activities using transient transfection assays in the human U2OS bone cell line with an estrogen response element linked to the luciferase reporter gene (ERE-tkLuc).

It has previously been shown (Paruthiyil S, et al. Cancer Res. 2004, 64, 423-8) that the growth promoting effects of estrogens are mediated by ERα, however the other known estrogen receptor, ERβ prevents breast cancer tumors in mice. These studies have prompted us to search for ERβ-selective compounds from Chinese herbs.

The root and rhizome of Rheum palmatum (Polygonaceae) were noted for their medicinal properties in the first systematic Chinese pharmacopeia, circa 200 AD. They have been in use ever since as purgatives for disorders like constipation, abdominal and gastric pain and fullness, as well as dysmenorrhea. They have also been used as antipyretics and as antiinflammatories to eliminate swelling and abscesses. They have also purported to remove blood clots and promote diuresis.

The following experiments demonstrate the estrogenic activity of anthraquinones 1 and 6.

Materials and Methods

General Experimental Procedures. ¹H, ¹³C, NMR spectra were measured, in pydridine-d5, using a Bruker AV-500 MHz spectrometer. A Shimadzu LC-10AT separation system, equipped with a Shimadzu SPD-10AV UV-Vis detector, using a Beckman (Fullerton, Calif.) Ultrasphere ODS column (250×10 mm, 5 μm) was used for HPLC analysis. Molecular weights for isolated compounds were determined at the Mass Spectrometry Facility, University of California Berkeley, using a ThermoFinnigan electrospray LCQ mass spectrometer in the positive and negative modes. Reversed-phase TLC analysis was performed on RP-18 F254 (Merck, Darmstadt, Germany) plates. Normal-phase column chromatography used silica gel 200-400 mesh, 60 Å, Aldrich Chemical Company, (St. Louis, Mo.). Prepsep, C18 sold phase extraction cartage were purchased from Fisher Scientific (Pittsburgh, Pa.). Chrysophanol, emodin, rhen were purchased from Sigma Chemical Company (St. Louis, Mo.). Aloe-emodin and frangulin A were purchased from ChromaDex (Santa Ana, Calif.).

Plant Material. The finely ground rhizome of Rheum palmatum (Da Huang) were purchased from Shen Nong Herbs, 1600 Shattuck Ave., Berkeley, Calif. 94703.

Extraction and Isolation. Finely ground R. palmatum rhizome (30 g) was extracted with 8:2 MeOH—H₂O for 1 h, 8 h, and overnight. The MeOH extracts were concentrated in vacuo to ca. 100 mL, resuspended in H₂O, and partitioned sequentially with hexane and EtOAc.

The EtOAc partition (200 mg) was applied to a solid phase extraction cartridge (5 g) and eluted with mixtures of A =10 mM ammonium acetate, B=MeCN (A-B 1:0, 75:25, 1:1, 25:75, and 0:1) to yield five fractions, A-E. Fraction C (250 mg) was chromatographed over Sephadex LH-20 (200×2.5 cm), and eluted with MeOH. Five combined fractions, A1-E1, were obtained by RP18 TLC analysis (6:4 10 mM ammonium acetate—MeCN). Fraction B1 was chromatographed on silica gel TLC (20×20 cm) developed with 8:2:0.1 hexane:EtOAc:TFA to yield octahydroxyanthraquinone (6), 10 mg, Rf=0.29. Fraction D (A-B 25:75) was chromatographed over silica gel (45×2.0 cm), and eluted with 8:2:0.1 hexane:EtOAc:TFA to yield chrysophanol (3), 5 mg, emodin (1), 10.3 mg, and aloe-emodin (2), 12 mg. Column chromatography was monitored by reversed-phase TLC, (1:1 10 mM ammonium acetate-MeCN).

Emodin (1): Yellow-orange powder; Positive ESIMS m/z 271 [M+H]+. ¹H and ¹³C NMR data are consistent with previously published data. The identification was further support by comparison of ¹H data and HPLC analysis with purchased standard (Sigma Chemical Company, St. Louis, Mo.).

Aloe-emodin (2): Yellow-orange powder; Negative ESIMS m/z 269 [M−H]−. ¹H and ¹³C NMR data are consistent with previously published data. The identification was further support by comparison of ¹H data and HPLC analysis with a purchased standard (ChromaDex Santa Ana, Calif.).

Chrysophanol (3): Yellow-orange powder; Negative ESIMS m/z 253 [M−H]−. ¹H and ¹³C NMR data are consistent with previously published data. The identification was further support by comparison of ¹H data and HPLC analysis with purchased standard (Sigma Chemical Company, St. Louis, Mo.).

Octahydroxyanthraquinone (6): Light yellow powder; Negative ESIMS m/z 335 [M−H]−. Data is consistent with previously published values.

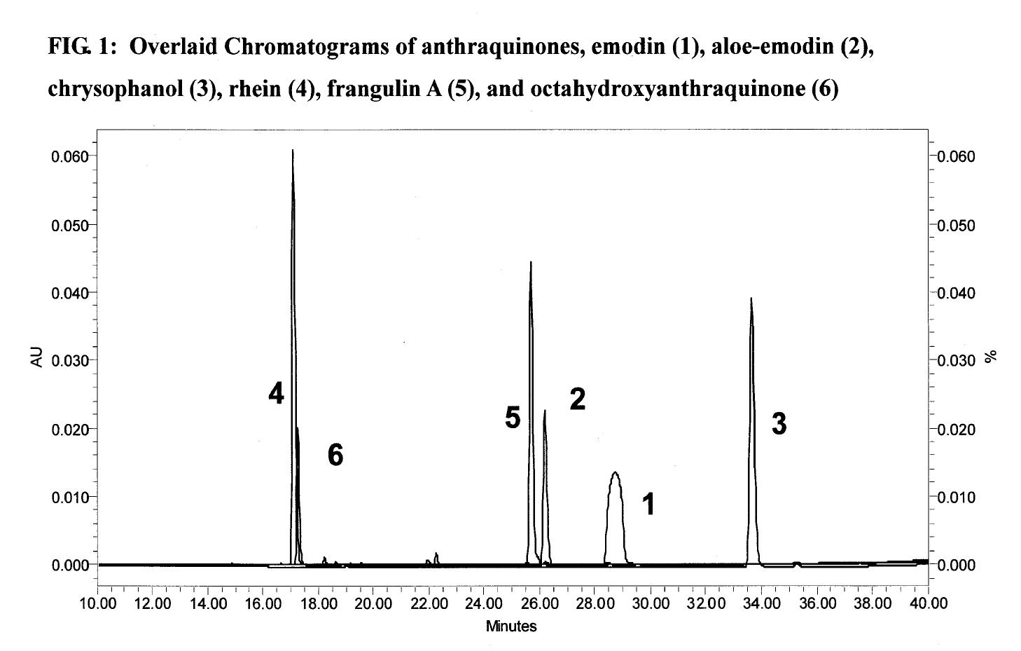

HPLC Analysis. The following solvent system was used, 10 mM ammonium acetate (A) and acetonitrile (B); the initial conditions were 90% A, 10% B. The initial condition was held for 4 minutes, then a linear gradient was initiated until minute 34; the final solvent mixture was 0% A, 100% B. The column was held at 100% B until minute 39 and then returned to initial conditions (90% A, 10% B) at minute 40. The column was reequilibrated for 10 minutes before the next injection. The flow rate was 1 mL/min for all HPLC experiments. A composite chromatogram is shown in FIG. 1.

Anthraquinones tested for their ERα and ERβ activities

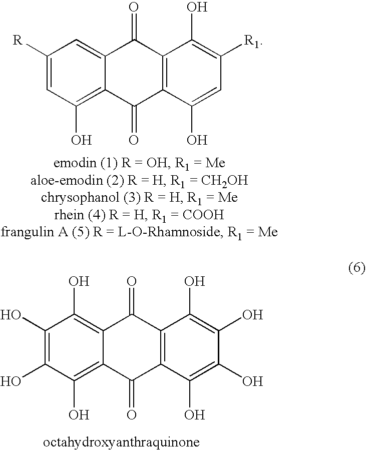

Results and Discussion

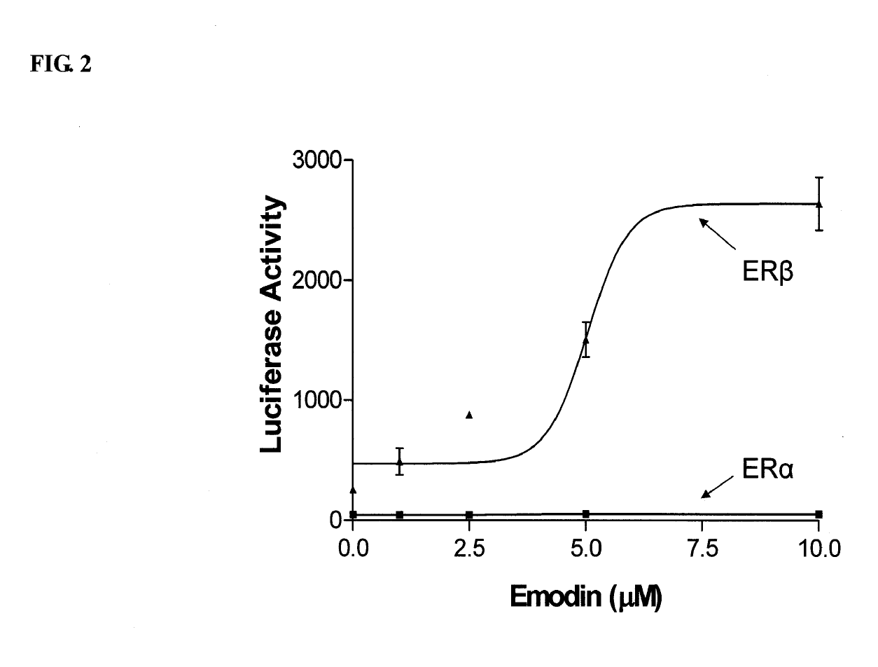

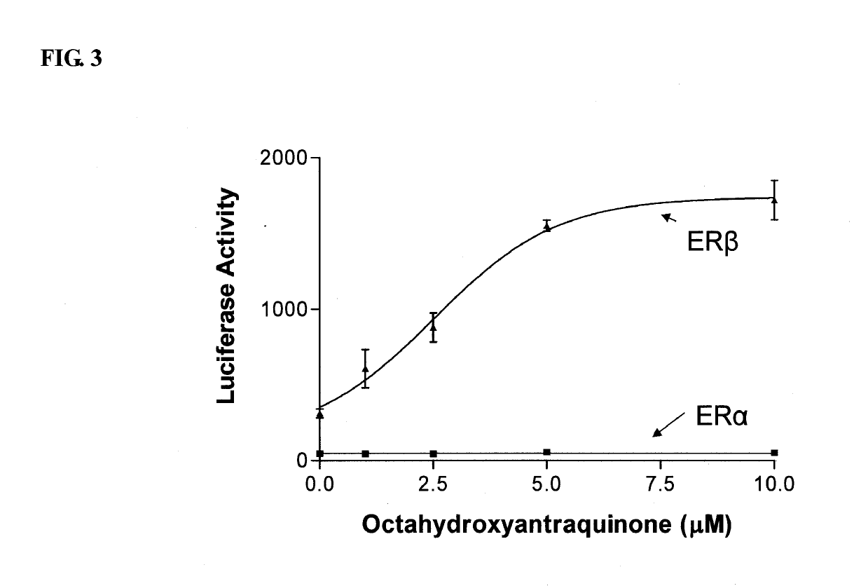

The anthraquinones, 1-6, identified from the rhizome of Rheum palmatum showed estrogenic activity. This activity was selective for estrogen receptor beta as demonstrated by activation of the ERE tkLuc. (FIG. 2, Emodin; FIG. 3, Octahydroxyanthraquinone). This activity was selective to the estrogen receptor beta because the compounds activated ERE tkLuc with ERβ. Emodin (1) showed greater activation than octahydroxyanthraquinone (6). Compare FIGS. 2, 3.

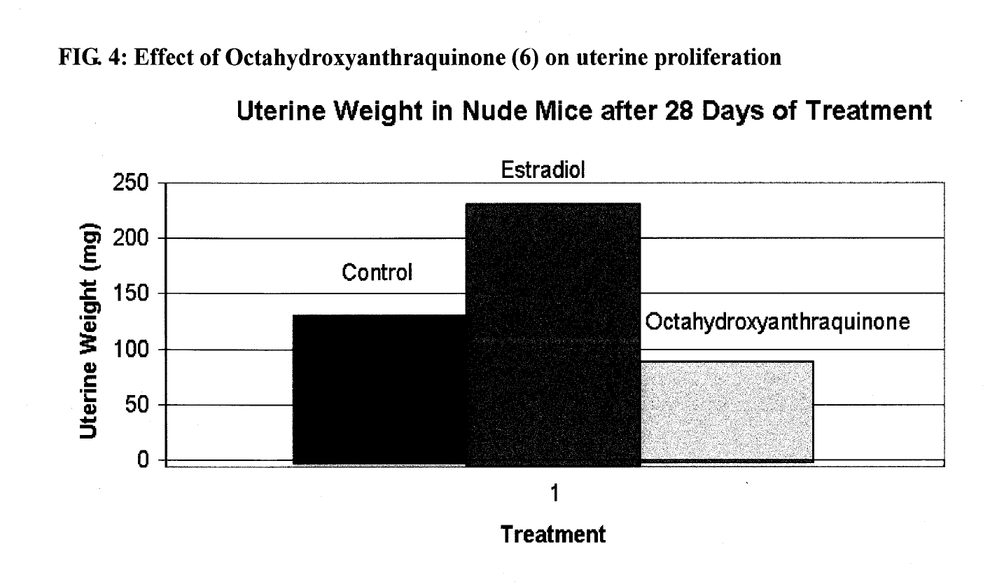

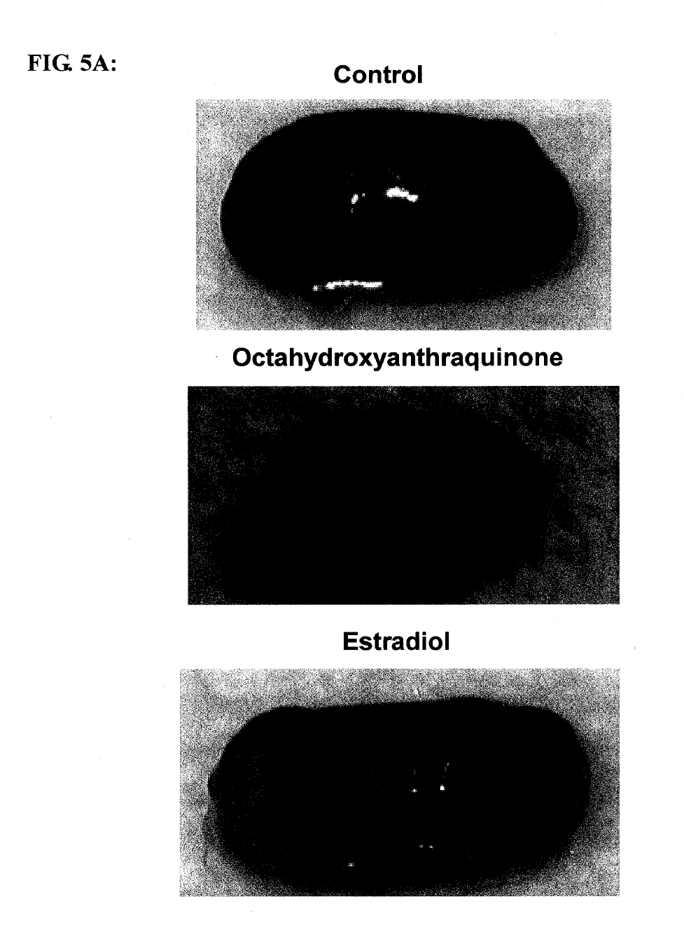

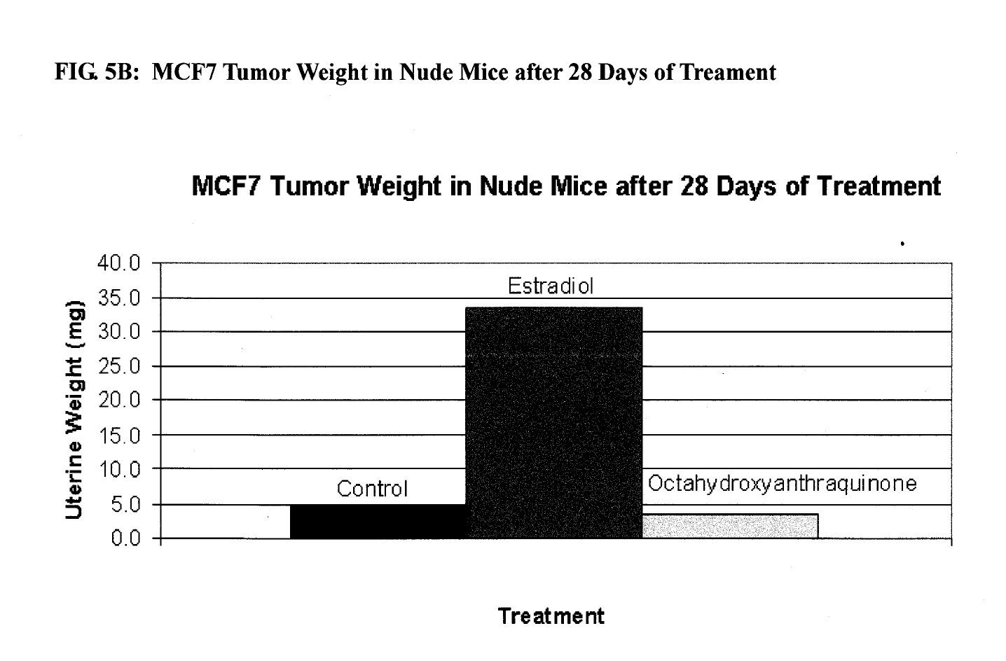

The effects of 6 were tested in vivo on breast cancer tumor formation and uterine proliferation. CD-1/Nude female mice, 40-50 days old at time of grafting were selected. Each mouse received 2 grafts per kidney, 4 grafts total with MCF-7 breast cancer cells in 25 μL collagen per graft. Compound 6 was delivered subcutaneously with a Alzet Osmotic pump filled with 200 μL of 6 for 28 days. The final dose of each compound was 360 μg/200 μL for the positive control estradiol and 2 mg/200 μL for octahydroxyanthraquinone (6). At the end of 28 days of treatment, all grafts and uteri were isolated, pictures taken, weighed, and fixed in formalin. When compared to controls, 6 does not increase breast cancer tumor formation (FIG. 4) nor does it increase uterine proliferation (FIGS. 5A. 5B).

### CONCLUSION

Since the results of the Women Health Initiative, hormone therapy trials have showed multiple significant health risks to menopausal women, therefore, safer estrogens are needed for the treatment of menopausal symptoms. Prior to clinical application of any new estrogens, safety information should be obtained concerning their potential risk in breast and uterine cancer promotion. In the current study we show that substances isolated from Rheum palmatum have estrogen receptor beta selective activity and these isolated compounds do not increase the risk of breast cancer tumor formation nor do they increase uterine proliferation in vivo. This confirms our prior studies showing the potential benefits of estrogen receptor beta selective compounds as potential estrogens for the treatment of menopausal symptoms.

### Example 2

### ER-Mediated Activation and Repression in the Presence of Emodin and Aloe-Emodin

Materials and Methods: Reagents. Phenol red-free Dulbecco's modified Eagle's/F-12 Coon's modification medium was obtained from Sigma. Biobrene was purchased from Applied Biosystems. The U937 cell line was obtained from American Type Culture Collection. Human recombinant TNF-α was obtained from R & D Systems.

Plasmid Construction. A PstI to AhaII fragment (−1044 to +93) from the human TNF-α gene, pLT, was cloned upstream of the luciferase cDNA. The 5′ deletions were constructed by using unique restriction sites, ApaI for the −125 deletion, and StyI for the -82 deletion. Three copies of the human TNF-α promoter fragment from −125 to −82 [TNF-responsive element (TNF-RE)] or one copy of the ERE from the frog vitellogenin A2 gene (vitA2-ERE, 5′-TCAGGTCACAGTGACCTGA-3′) were ligated upstream of −32 to +45 herpes simplex thymidine kinase (TK) promoter linked to luciferase (TNF-RE tkLuc, and ERE TKLuc, respectively). ERβ mutants were created with QuikChange site-directed mutagenesis kits (Stratagene), by using oligonucleotides containing the mutation. The mutants were sequenced with Sequenase kits (Amersham Pharmacia) to verify the presence of the mutation.

Cell Culture, Transfection, and Luciferase Assays - U937 (human monocyte), U20S (human osteosarcoma), MDA-MB-435 (human metastatic breast cancer), and MCF-7 (human breast cancer) cells were obtained from the cell culture facility at the University of California, San Francisco. U937 cells were maintained as described previously, whereas U2OS, MDA-MB-435, and MCF-7 cells were maintained and subcultured in phenol red-free Dulbecco's modified Eagle's medium/F-12 media containing 5% fetal bovine serum, 2 mM glutamine, 50 units/ml penicillin, and 50 μg/ml streptomycin. For experiments, cells were collected, transferred to a cuvette, and then electroporated with a Bio-Rad gene pulser as described previously using 3 μg of reporter plasmid and 1 μg of ERα or ERβ expression vectors. After electroporation, the cells were resuspended in media and plated at 1 ml/dish in 12-well multiplates. The cells were treated with E₂, genistein, daidzein, or biochanin A (Sigma-Aldrich) 3 hr prior to exposure to 5 ng/ml TNF-α (R & D Systems) for 24 hr at 37° C. Cells were solubilized with 200 μL of 1× lysis buffer, and luciferase activity was determined using a commercially available kit (Promega). The concentration of hormone required to produce a half-maximal induction (EC₅₀) or inhibition (IC50) of luciferase activity was calculated with the Prism curve-fitting program (Graph Pad Software, version 2.0b). For proliferation studies, parental MCF-7 cells were subcloned at 1 cell/well in the presence of 0.1 nM E₂, and the fastest growing clone was selected for experiments. These cells expressed exclusively ERα as determined by reverse transcription polymerase chain reaction (RT-PCR). The cells were plated in duplicate at a density of 25,000 cells/35-mm plate in tissue culture medium containing 3% stripped fetal bovine serum. One day after plating they were treated with increasing concentrations of Emodin or Aloe-Emodin. The medium was changed every other day, and Emodin or Aloe-Emodin was added to the medium. After 8 days the cells were counted with a Coulter counter. All experiments presented in the figures were performed at least three times, and the data were similar between experiments.

Emodin (BNER 1109) and Aloe-Emodin (BNER 1110) were prepared as described in Example 1, above.

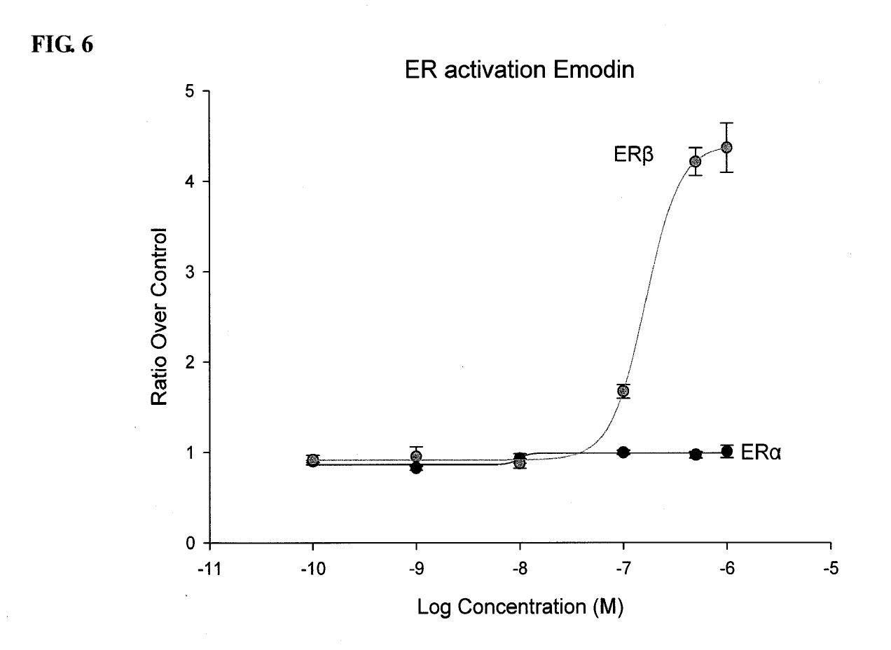

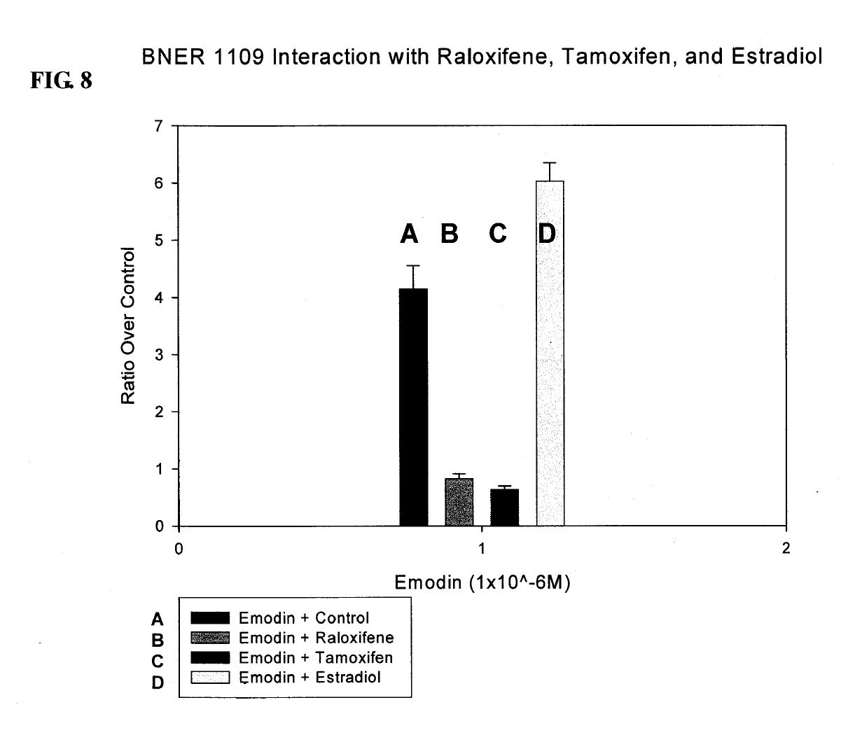

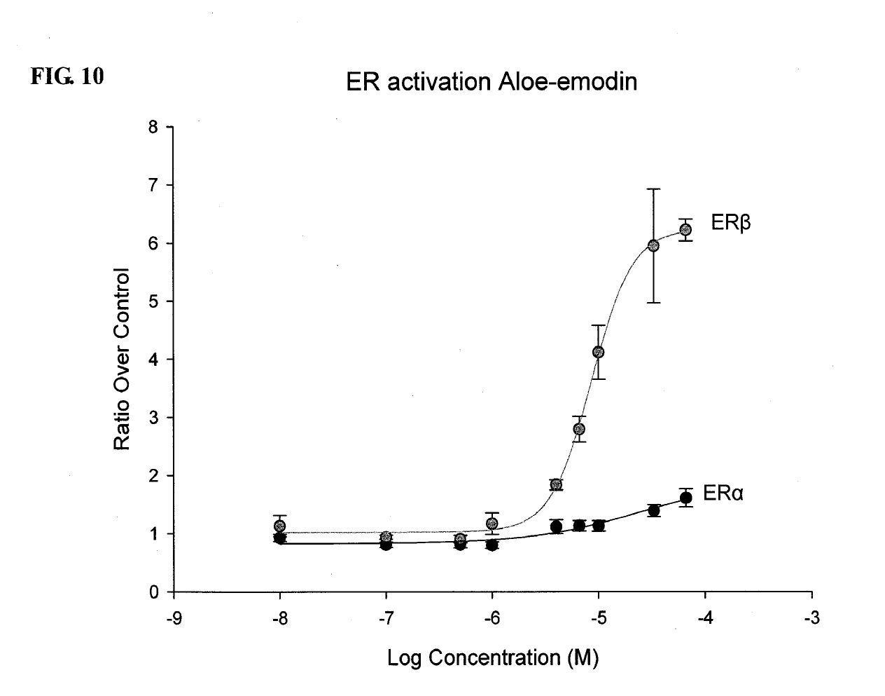

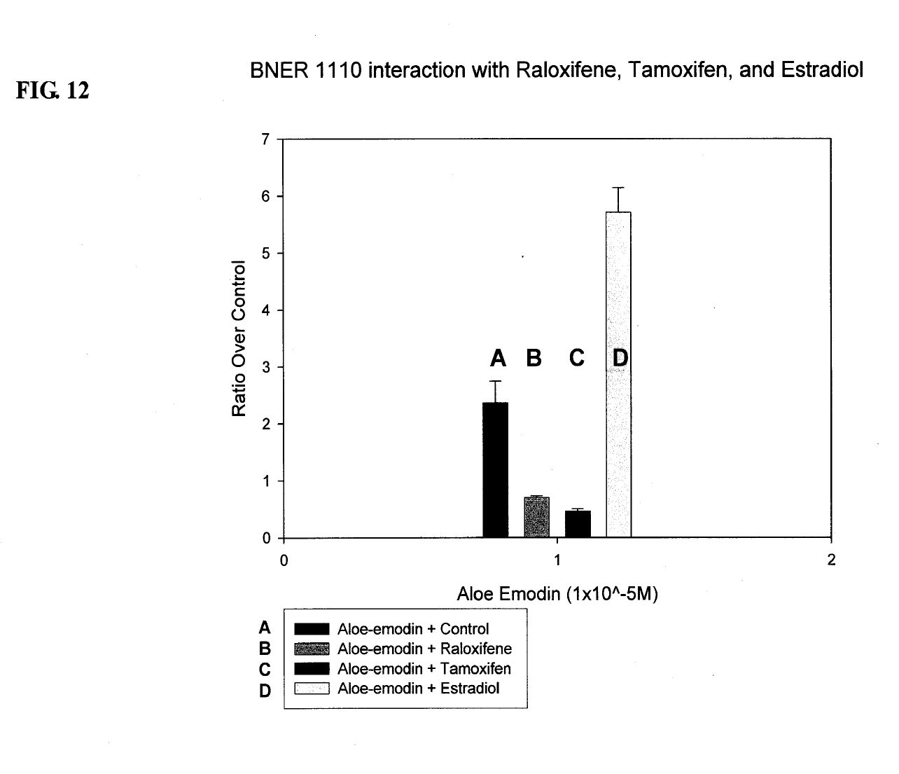

Results: Selective estrogen receptor modulating activity in U2OS Bone cells was measured using luciferase assays. U2OS osteosarcoma cells were cotransfected with a classic ERE upstream of a minimal thymidine kinase (tk) promoter (ERE-tk-Luc) and expression vectors for human ERα or ERβ. Emodin and Aloe Emodin activated the transfected ERE-tk-Luc gene in the presence of ERβ, but not significantly in the presence of ERα. (FIGS. 6, 10). Both Emodin and Aloe-Emodin stimulate expression of Luciferase by and ERE-tk-Luciferase transcript in the presence of ERβ; this effect is attenuated by co-administration with the extract of the selective ERβ antagonists Raloxifene and Tamoxifen. FIGS. 8, 12. These results indicate Emodin and Aloe-Emodin each activate ERE-tk-Luc by directly interacting with ERβ.

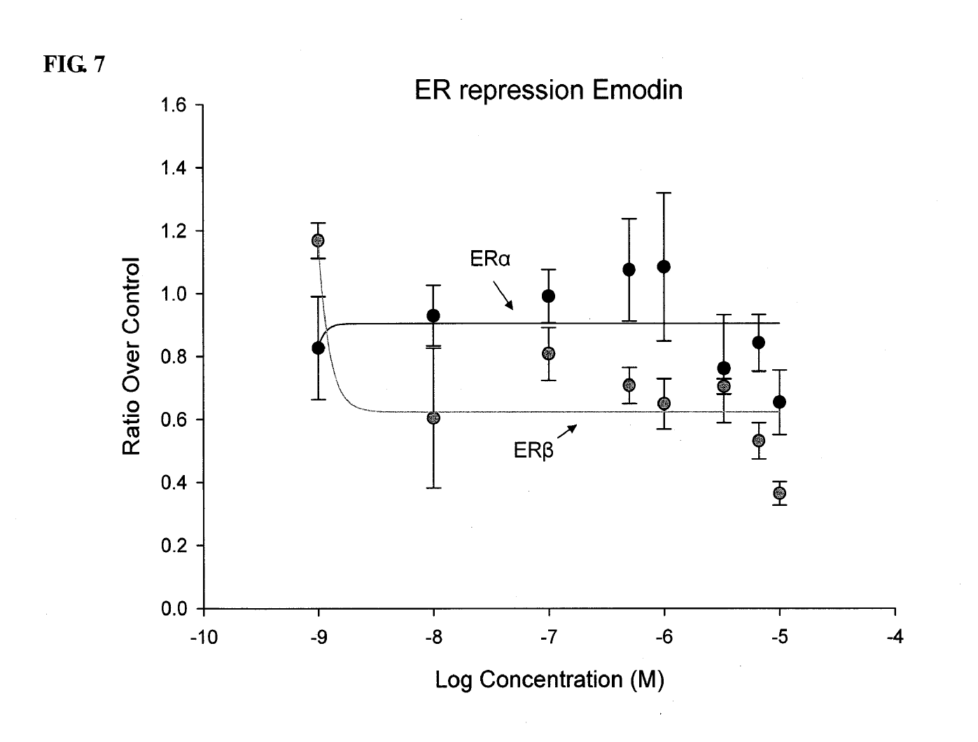

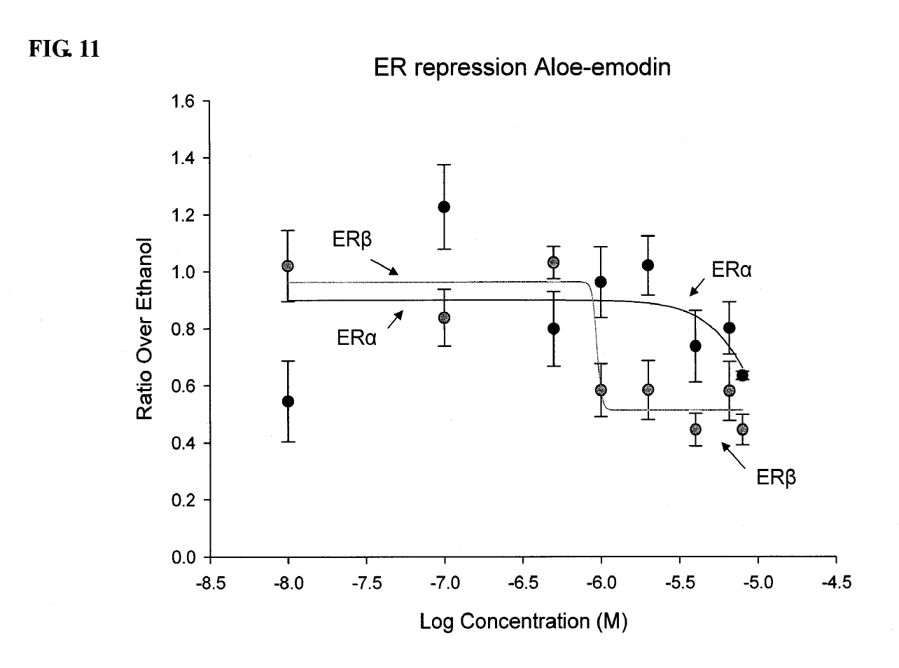

To investigate the effects Emodin and Aloe-Emodin on transcriptional repression, the −125 to −82 region of the TNF-α promoter (TNF-α-responsive element, (TNF-RE)) was used because this region mediates TNF-α activation and E₂ repression. E₂ produces a profound repression of TNF-α activation of the TNF-RE upstream of a minimal tk promoter (TNF-RE tkLuc) with either transfected ERα or ERα in U2OS cells. (Data not shown). E₂ can abolish TNF-α activity on ERβ (100% repression) but not on ERα (73.3% repression). Emodin produced repression of TNF-α activation of TNF-RE in the presence of ERβ, but not ERα (FIG. 7). In contrast, Aloe-Emodin produced repression of the TNF-α activation of TNF-RE in the presence of both ERβ and ERα (FIG. 11). These results indicate that Emodin and Aloe-Emodin repress TNF-α activation through TNF RE-tk-Luc by directly interacting with ERβ and/or ERα.

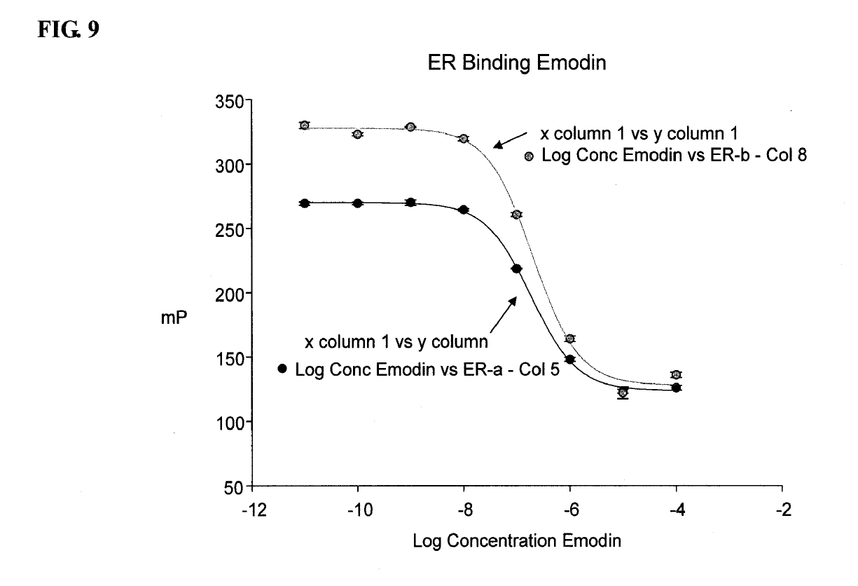

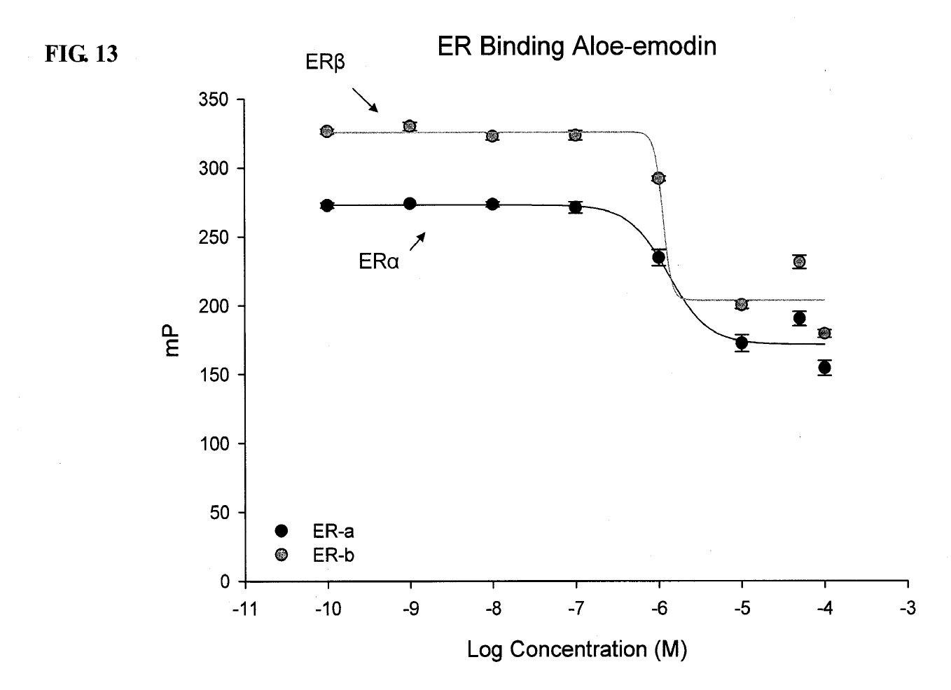

Interestingly, the ERβ-binding curve for Emodin is very similar to the ERα-binding curve. (FIG. 9) The same is true for the ERβ-binding curve for Aloe-Emodin as compared to the ERα-binding curve. (FIG. 13).

### Example 3

### Open Label Increasing Dose, Dosing Study

The following protocol is carried out in order to determine the maximum tolerated dose for a pharmaceutical composition comprising one or more compounds of formula II:

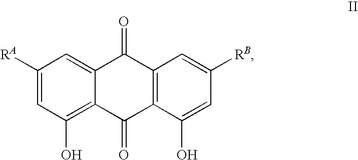

wherein either (1) R^{A} is OH and R^{B} is CH₃; (2) R^{A} is H and R^{B} is CH₂OH; (3) R^{A} is H and R^{B} is CH₃.

Study Drug comprises 1 mg (week 1), 10 mg (week 2), 100 mg (week 3) or 1000 mg (week 4) of a pharmaceutical composition comprising one or more compounds of formula II. (Hereinafter the pharmaceutical composition comprising one or more compounds of formula II may be referred to as “Study Drug”). The dose may be split between two or more gelatin capsules if necessary. Normal, healthy volunteers of age 18 to 60 are administered 1 mg per day of Study Drug for week 1, 10 mg per day of Study Drug for week 2, 100 mg per day of study drug for week 3 and 1000 mg per day of Study Drug for week 4. Subjects are monitored for appearance of any adverse events. At any time, if a subject appears to not tolerate the current dose, the attending medical staff will note such intolerance. The maximum tolerated dose will be considered the highest dose at which each of the subjects tolerates the dose, or, if no subject experiences intolerance, 1000 mg of the Study Drug per day.

### Example 4

### Open Label, Increasing Dose, Dosing Study

The following protocol is carried out in order to determine the maximum tolerated dose for a pharmaceutical composition comprising a compound 6 (octahydroxyanthraquinone):

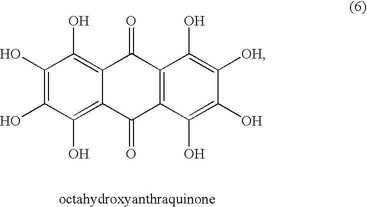

Study Drug comprises 1 mg (week 1), 10 mg (week 2), 100 mg (week 3) or 1000 mg (week 4) of a pharmaceutical composition comprising 6. (Hereinafter the pharmaceutical composition comprising 6 may be referred to as “Study Drug”). The dose may be split between two or more gelatin capsules if necessary. Normal, healthy volunteers of age 18 to 60 are administered 1 mg per day of Study Drug for week 1, 10 mg per day of Study Drug for week 2, 100 mg per day of study drug for week 3 and 1000 mg per day of Study Drug for week 4. Subjects are monitored for appearance of any adverse events. At any time, if a subject appears to not tolerate the current dose, the attending medical staff will note such intolerance. The maximum tolerated dose will be considered the highest dose at which each of the subjects tolerates the dose, or, if no subject experiences intolerance, 1000 mg of the Study Drug per day.

Although the invention has been illustrated with reference to certain embodiments and examples, the person having skill in the art will recognize that other embodiments are envisioned within the scope of the present invention.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A pharmaceutical composition, comprising an amount of at least one isolated and purified compound of formula I or 6: 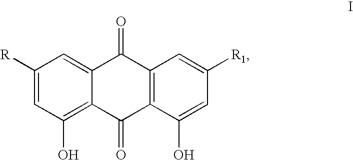
where R is H or OH; and
R¹ is C₁-C₄ alkyl (methyl, ethyl, i-propyl, n-propyl, i-butyl, n-butyl, s-butyl or t-butyl) or CH₂OH; or
octahydroxyanthraquinone (formula 6): 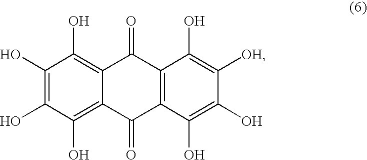
wherein the amount of the at least one compound of formula I and/or octahydroxyanthraquinone is sufficient to modulate estrogen receptor beta (ERβ) in a multicellular organism.

2. (canceled)

3. The composition of claim 1, wherein at least one compound of formula I is selected from compounds 1, 2 and 3, wherein the compounds 1, 2 and 3 are of formula II: 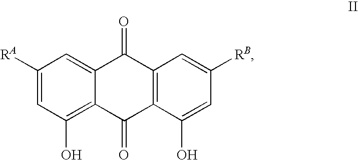
wherein for compound (1) R^{A} is OH and R^{B} is CH₃; for compound (2) R^{A} is H and R^{B} is CH₂OH; and for compound (3) R^{A} is H and R^{B} is CH₃; and the compound of formula (6) is:

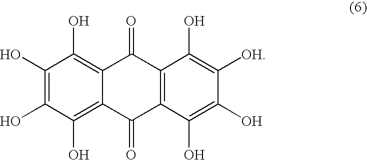

4. The composition of claim 3, comprising: (a) two or more of (1), (2), (3) and/or (6); (b) three or more of (1), (2), (3) and/or (6); (c) all four of (1), (2), (3) and/or (6); or (d) compound (6) and two or more of (1), (2), and/or (3).

5-21. (canceled)

22. A method of eliciting an estrogenic effect, comprising administering to a subject an estrogenically effective amount of a composition comprising at least one isolated and purified compound of formula I or 6: 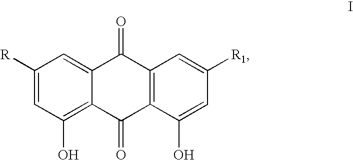
where R is H or OH; and
R¹ is C₁-C₄ alkyl or CH₂OH; and compound 6 has the formula: 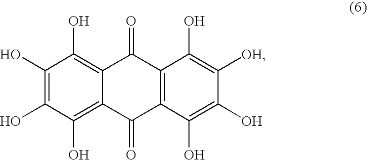
wherein the amount administered to the patient is sufficient to modulate (e.g. agonize) estrogen receptor beta (ERβ) in a multicellular organism.

23. (canceled)

24. The method of claim 22, wherein at least one compound of formula I is selected from compounds 1, 2 and 3, wherein the compounds 1, 2 and 3 are of formula II: 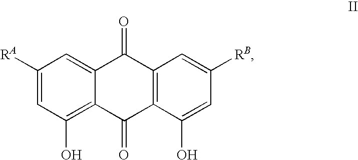
wherein for compound (1) R^{A} is OH and R^{B} is CH₃; for compound (2) R^{A} is H and R^{B} s CH₂OH; and for compound (3) R^{A} is H and R^{B} is CH₃; and the compound of formula (6) is:

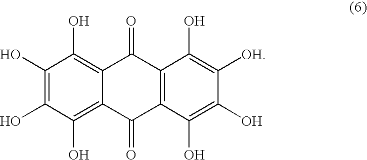

25. The method of claim 22, wherein the composition comprises (a) two or more of (1), (2), (3) and/or (6); (b) three or more of (1), (2), (3) and/or (6); all four of (1). (2), (3) and/or (6); or (d) (6) and two or more of (1), (2) and/or (3).

26-29. (canceled)

30. The method of claim 22, wherein the composition is substantially free of at least one of rhein and frangulin A.

31. The method of claim 22, wherein the composition is substantially free of both of rhein and frangulin A.

32. The method of claim 22, wherein the estrogenic effect includes at least one effect selected from the group consisting of: treating or preventing at least one climacteric symptom; treating or preventing osteoporosis; treating or preventing uterine cancer; treating or preventing breast cancer; treating or preventing cervical cancer; treating or preventing cancer of the ovary; and treating or preventing cardiovascular disease.

33. The method of claim 32, wherein the estrogenic effect includes treating or preventing at least one climacteric symptom selected from the group consisting of treating or preventing hot flashes, insomnia, vaginal dryness, decreased libido, urinary incontinence and depression.

34. The method of claim 32, wherein the estrogenic effect includes treating or preventing osteoporosis.

35. The method of claim 32, wherein the estrogenic effect includes treating or preventing hot flashes.

36. The method of claim 32, wherein the estrogenic effect includes treating or preventing uterine cancer or breast cancer.

37. The method of claim 22, wherein the estrogenic effect does not include increasing the risk of mammary hyperplasia, mammary tumor, uterine hyperplasia, uterine tumor, cervical hyperplasia, cervical tumor, ovarian hyperplasia, ovarian tumor, fallopian tube hyperplasia, fallopian tube tumor.

38. The method of claim 22, wherein the estrogenic effect includes decreasing the risk of mammary hyperplasia, mammary tumor, uterine hyperplasia, uterine tumor, cervical hyperplasia, cervical tumor, ovarian hyperplasia, ovarian tumor, fallopian tube hyperplasia, fallopian tube tumor.

39. A method of activating a gene under control of an estrogen response element, comprising administering to a cell having an estrogen response element operatively linked to the gene and an estrogen receptor an amount of a composition of claim 1 sufficient to activate said gene.

40-52. (canceled)

53. A method of repressing expression of a TNF RE-controlled gene, comprising administering to a cell comprising a gene under control of a TNF response element and an estrogen receptor an amount of a composition of claim 1 effective to repress said TNF RE-controlled gene.

54-67. (canceled)

68. A process of isolating emodin from Rheum palmatum, comprising:
(a) contacting optionally wholly or partially comminuted rhizome of Rheum palmatum with aqueous methanol;
(b) separating the rhizome from the aqueous methanol to form an aqueous methanol extract;
(c) evaporating methanol from the aqueous methanol extract to form a concentrate;
(d) adding water to the concentrate to form an aqueous slurry;
(e) contacting the aqueous slurry with hexane and separating the hexane from the aqueous slurry;
(f) contacting the aqueous slurry with ethyl acetate;
(g) separating the ethyl acetate from the aqueous slurry;
(h) applying the ethyl acetate to a solid phase extraction substrate;
(i) serially eluting the extraction substrate with serial aliquots of elution solvents (A)-(D):
(A) aqueous ammonium acetate,
(B) aqueous ammonium acetate and acetonitrile in a ratio of about 4:1 to about 2.5:1;
(C) aqueous ammonium acetate and acetonitrile in a ratio of about 0.9:1 to about 1.1:1; and
(D) aqueous ammonium acetate and acetonitrile in a ratio of about 1:2.5 to about 1:4;
(j) collecting an eluate of (D) and applying the collected eluate to a silica gel;
(k) contacting the silica gel with a mixture of hexane, ethyl acetate and trifluoroacetic acid; and
(l) collecting a fraction containing emodin from the silica gel.

69. The process of claim 68, wherein one or more of the following applies:
(i) elution solvent (B) contains about 10 mM ammonium acetate (aqueous) and acetonitrile in a ratio of about 3:1 (v/v);
(ii) elution solvent (C) contains about 10 mM ammonium acetate (aqueous) and acetonitrile in a ratio of about 1:1 (v/v);
(iii) elution solvent (D) contains about 10 mM ammonium acetate (aqueous) and acetonitrile in a ratio of about 1:3 (v/v);
(iv) the solid phase extraction substrate in (h) is a reverse phase extraction substrate in a column or cartridge;
(v) the silica gel in (j) is on a silica gel thin layer chromatography (TLC) plate; and/or (vi) mixture of hexane, ethyl acetate and trifluoroacetic acid in (k) is in a ratio of about 8:2:0.1.

70. A process of isolating aloe-emodin from Rheum palmatum, comprising:
(a) contacting optionally wholly or partially comminuted rhizome of Rheum palmatum with aqueous methanol;
(b) separating the rhizome from the aqueous methanol to form an aqueous methanol extract;
(c) evaporating methanol from the aqueous methanol extract to form a concentrate;
(d) adding water to the concentrate to form an aqueous slurry;
(e) contacting the aqueous slurry with hexane and separating the hexane from the aqueous slurry;
(f) contacting the aqueous slurry with ethyl acetate;
(g) separating the ethyl acetate from the aqueous slurry;
(h) applying the ethyl acetate to a solid phase extraction substrate;
(i) eluting the extraction substrate with serial aliquots of extraction solvents (A)-(D):
(A) aqueous ammonium acetate,
(B) aqueous ammonium acetate and acetonitrile in a ratio of about 4:1 to about 2.5:1;
(C) aqueous ammonium acetate and acetonitrile in a ratio of about 0.9:1 to about 1.1:1; and
(D) aqueous ammonium acetate and acetonitrile in a ratio of about 1:2.5 to about 1:4;
(j) collecting an eluate of (D) and applying the collected eluate to a silica gel;
(k) contacting the silica gel with a mixture of hexane, ethyl acetate and trifluoroacetic acid in a ratio of about 8:2:0.1; and
(l) collecting a fraction containing aloe-emodin from the silica gel.

71. The process of claim 70, wherein one or more of the following applies:
(i) elution solvent (B) contains about 10 mM ammonium acetate (aqueous) and acetonitrile in a ratio of about 3:1 (v/v);
(ii) elution solvent (C) contains about 10 mM ammonium acetate (aqueous) and acetonitrile in a ratio of about 1:1 (v/v);
(iii) elution solvent (D) contains about 10 mM ammonium acetate (aqueous) and acetonitrile in a ratio of about 1:3 (v/v);
(iv) the solid phase extraction substrate in (h) is a reverse phase extraction substrate in a column or cartridge;
(v) the silica gel in (j) is on a silica gel thin layer chromatography (TLC) plate; and/or
(vi) the mixture of hexane, ethyl acetate and trifluoroacetic acid in (k) is in a ratio of about 8:2:0.1.

72. A process of isolating chrysophanol from Rheum palmatum, comprising:
(a) contacting optionally wholly or partially comminuted rhizome of Rheum palmatum with aqueous methanol;
(b) separating the rhizome from the aqueous methanol to form an aqueous methanol extract;
(c) evaporating methanol from the aqueous methanol extract to form a concentrate;
(d) adding water to the concentrate to form an aqueous slurry;
(e) contacting the aqueous slurry with hexane and separating the hexane from the aqueous slurry;
(f) contacting the aqueous slurry with ethyl acetate;
(g) separating the ethyl acetate from the aqueous slurry;
(h) applying the ethyl acetate to a solid phase extraction substrate;
(i) eluting the extraction substrate with serial aliquots of extraction solvents (A)-(D):
(A) aqueous ammonium acetate,
(B) aqueous ammonium acetate and acetonitrile in a ratio of about 4:1 to about 2.5:1;
(C) aqueous ammonium acetate and acetonitrile in a ratio of about 0.9:1 to about 1.1:1; and
(D) aqueous ammonium acetate and acetonitrile in a ratio of about 1:2.5 to about 1:4;
(j) collecting an eluate of (D) and applying the collected eluate to a silica gel;
(k) contacting the silica gel with a mixture of hexane, ethyl acetate and trifluoroacetic acid in a ratio of about 8:2:0.1; and
(l) collecting a fraction from the silica gel containing chrysophanol.

73. The process of claim 72, wherein one or more of the following applies:
(i) elution solvent (B) contains about 10 mM ammonium acetate (aqueous) and acetonitrile in a ratio of about 3:1 (v/v);
(ii) elution solvent (C) contains about 10 mM ammonium acetate (aqueous) and acetonitrile in a ratio of about 1:1 (v/v);
(iii) elution solvent (D) contains about 10 mM ammonium acetate (aqueous) and acetonitrile in a ratio of about 1:3 (v/v);
(iv) the solid phase extraction substrate in (h) is a reverse phase extraction substrate in a column or cartridge;
(v) the silica gel in 0) is on a silica gel thin layer chromatography (TLC) plate; and/or (vi) the mixture of hexane, ethyl acetate and trifluoroacetic acid in (k) is in a ratio of about 8:2:0.1.

74. A process of isolating octahydroxyanthraquinone from Rheum palmatum, comprising:
(a) contacting optionally wholly or partially comminuted rhizome of Rheum palmatum with aqueous methanol;
(b) separating the rhizome from the aqueous methanol to form an aqueous methanol extract;
(c) evaporating methanol from the aqueous methanol extract to form a concentrate;
(d) adding water to the concentrate to form an aqueous slurry;
(e) contacting the aqueous slurry with hexane and separating the hexane from the aqueous slurry;
(f) contacting the aqueous slurry with ethyl acetate;
(g) separating the ethyl acetate from the aqueous slurry;
(h) applying the ethyl acetate to a solid phase extraction cartridge;
(i) eluting the extraction cartridge with serial aliquots of
(A) aqueous ammonium acetate,
(B) aqueous ammonium acetate and acetonitrile in a ratio of about 4:1 to about 2.5:1; and
(C) aqueous ammonium acetate and acetonitrile in a ratio of about 0.9:1 to about 1.1:1;
(j) collecting an eluate of (C) and applying the collected eluate to a resin separation substrate, eluting with a lower alcohol (such as ethanol or methanol) and collecting an octahydroxyanthraquinone-containing fraction;
(l) applying the collected fraction to a reverse phase separation substrate and fractionating with a mixture of ammonium acetate and acetonitrile in a ratio of about 5:5 to about 7:3;
(m) collecting a fraction containing octahydroxyanthraquinone and applying the obtained fraction to a silica gel;
(n) collecting a fraction from the developed silica gel containing octahydroxyanthraquinone.

75. The process of claim 74, wherein one or more of the following applies:
(i) elution solvent (B) contains about 10 mM ammonium acetate (aqueous) and acetonitrile in a ratio of about 3:1 (v/v);
(ii) elution solvent (C) contains about 10 mM ammonium acetate (aqueous) and acetonitrile in a ratio of about 1:1 (v/v);
(iii) the solid phase extraction substrate in (h) is a reverse phase extraction substrate in a column or cartridge;
(iv) the silica gel in (j) is on a silica gel thin layer chromatography (TLC) plate; and/or
(v) the mixture of hexane, ethyl acetate and trifluoroacetic acid in (k) is in a ratio of about 8:2:0.1.
